(19) United States
(12) Patent Application Publication
(10) Pub. No.: US 2012/0053227 A1
(43) Pub. Date: Mar. 1, 2012

(54) miR-33 INHIBITORS AND USES THEREOF

(75) Inventors: Carlos Fernández-Hernando, New York, NY (US); Kathryn J. Moore, Westfield, NJ (US); Yajaira Suárez, New York, NY (US); Katey J. Rayner, New York, NY (US)

(73) Assignee: New York University

(21) Appl. No.: 13/219,347

(22) Filed: Aug. 26, 2011

Related U.S. Application Data
(60) Provisional application No. 61/377,789, filed on Aug. 27, 2010.
(60) Provisional application No. 61/412,720, filed on Nov. 11, 2010.
(60) Provisional application No. 61/480,212, filed on Apr. 28, 2011.

(51) Int. Cl. A61K 31/7115; A61P 9/10; A61P 3/00

(52) U.S. Cl. 514/44 A

(57) ABSTRACT

The miRNA miR-33 is shown to inhibit the expression of carnitine O-octaniltransferase (CROT), Carnitine palmitoyltransferase 1A (CPT1a) and hydroxyacyl-CoA-dehydrogenase (HADHB), reduce fatty acid oxidation in hepatic cells, and target the insulin receptor substrate 2 (IRS-2) independent of its ability to elevating plasma high density lipoprotein (HDL) levels. MiR-33 inhibitors are also shown to increase cholesterol efflux from peripheral cells, such as cholesterol-laden macrophages present in atherosclerotic plaques. Compositions and methods are therefore provided for treating or preventing metabolic syndrome and atherosclerosis using miR-33 inhibitors. The miR-33 inhibitors are preferably antagomirs having a single-stranded nucleic acid sequence that is complementary to at least 12 contiguous nucleotides in miR-33 and therefore forms a duplex with miR-33 under physiological conditions.

### CROSS REFERENCE TO RELATED APPLICATIONS

This application claims benefit of U.S. Provisional Application No. 61/377,789, filed Aug. 27, 2010, U.S. Provisional Application No. 61/412,720, filed Nov. 11, 2010, and U.S. Provisional Application No. 61/480,212, filed Apr. 28, 2011, which are hereby incorporated herein by reference in their entirety.

### STATEMENT REGARDING FEDERALLY SPONSORED RESEARCH OR DEVELOPMENT

This invention was made with Government Support under Grant Nos. R01AG02055 and R01HL108182 awarded to Kathryn J. Moore by the National Institutes of Health; Grant No. R01HL074136 awarded to Michael L. Fitzgerald by the National Institutes of Health; Grant No. R01HL084312 awarded to Edward A. Fisher by the National Institutes of Health; Grant No. 1P30HL101270-01 and R01HL16063 awarded to Carlos Fernández Hernando by the National Institutes of Health; and Grant No. HL088528 to Ryan E. Temel by the National Institutes of Health. The Government has certain rights in the invention.

### REFERENCE TO SEQUENCE LISTING

The Sequence Listing submitted Aug. 26, 2011, as a text file named “NYUMC_HER04_{—}01_ST25.txt,” created on Aug. 26, 2011, and having a size of 8,871 bytes is hereby incorporated by reference.

### FIELD OF THE INVENTION

The invention is generally related to the field of molecular biology, more specifically to the field of antagomirs and their use in inhibiting microRNAs for treating metabolic syndrome and atherosclerosis.

### BACKGROUND OF THE INVENTION

Metabolic syndrome is a collection of health disorders or risks that increase the chance of developing heart disease, stroke, and diabetes. The condition is also known by other names, including Syndrome X, insulin resistance syndrome, and dysmetabolic syndrome. Metabolic syndrome can include any of a variety of underlying metabolic phenotypes, including insulin resistance and obesity predisposition phenotypes, low HDL and hypertriglyceridemia.

Metabolic syndrome is extremely common, particularly in the United States, where roughly 50 million people are thought to have the disorder. Roughly one in five Americans has metabolic syndrome. The number of people with metabolic syndrome increases with age, affecting more than 40 percent of people in their 60s and 70s. The underlying causes of metabolic syndrome are, in many respects, quite unclear-though certain effects of the disorder such as obesity and lack of physical activity are often causal in nature as well. Given inheritance patterns for the disorder, there also appear to be genetic factors that underlie the syndrome.

People diagnosed with the metabolic syndrome are at increased risk of coronary heart disease, plaque buildups in artery walls, and type 2 diabetes. In addition, metabolic syndrome increases the risk for Alzheimer disease.

Currently, first-line therapy for individuals with metabolic syndrome is primarily directed at lifestyle changes such as dietary changes and weight reduction. Drug treatment to reduce insulin resistance may also be used, as well as other therapies targeted to the individual components of metabolic syndrome. However, since metabolic syndrome is usually associated with several clinical situations, a medicament having hypoglycemic, hypolipidemic effects as well as improving insulin resistance is needed show significant therapeutic effects for metabolic syndrome.

Atherosclerosis (also known as arteriosclerotic vascular disease or ASVD) is a condition in which an artery wall thickens as the result of a build-up of fatty materials such as cholesterol. The excessive uptake of lipoproteins by cells in the arterial wall gives rise to cholesterol-loaded cells known as foam cells, which form the hallmark of atherosclerotic plaques. The efflux of cholesterol from foam cells is believed to be mediated by high-density lipoprotein (HDL) or its apolipoproteins and may represent a crucial step in the prevention or reversal of atherosclerosis. For example, macrophage cholesterol efflux has been proposed as the initial step in the reverse cholesterol transport (RCT). However, due to the multi-factorial nature of plaque formation, there has been insufficient progress in promoting RCT.

It is therefore an object of the invention to provide therapeutic compositions and method for treating metabolic syndrome in a subject.

It is therefore an object of the invention to provide therapeutic compositions and method for decreasing serum triglycerides.

It is a further object of the invention to provide therapeutic compositions and method for increasing cholesterol efflux from peripheral cells present in atherosclerotic plaques.

It is therefore an object of the invention to provide therapeutic compositions and method for treating or preventing atherosclerosis.

### SUMMARY OF THE INVENTION

MicroRNAs (miRNAs) are endogenous double stranded RNAs about 20 to 25 nucleotides in length that serve as posttranscriptional regulators of physiological processes by binding to complementary target sites in the 3′ untranslated regions (3′UTR) of mRNAs and causing translational repression and/or mRNA destabilization. A single miRNA can have multiple targets, potentially providing simultaneous regulation of genes involved in a physiological pathway. A role for miRNA miR-33 in the maintenance of cholesterol homeostasis had been identified. miR-33 is shown to regulate both HDL biogenesis in the liver and cellular cholesterol efflux. This activity of miR-33 is due at least in part to its inhibition of the ATP-binding cassette ABCA1. miR-33 binds the 3′UTR region of ABCA1 and inhibits its expression. MiR-33 is also shown to inhibit the expression of carnitine O-octaniltransferase (CROT), Carnitine palmitoyltransferase 1A (CPT1a) and hydroxyacyl-CoA-dehydrogenase (HADHB), reduce fatty acid oxidation in hepatic cells, and target the insulin receptor substrate 2 (IRS-2). In addition, miR-33 has been shown to inhibit the expression of AMP-activated protein kinase (PRKAA1). In some embodiments, miR-33 has these effects independent of its ability to elevating plasma high density lipoprotein (HDL) levels. MiR-33 inhibitors increase serum HDL levels, increase cholesterol efflux from peripheral macrophages, and decrease atherosclerotic plaques. In addition, miR-33 inhibitors decrease SREBP-1 expression, decrease genes involved in fatty acid synthesis (e.g., FAS, ACACA, ACLY), and reduce plasma VLDL triglycerides.

Therefore, compositions and methods for treating metabolic syndrome are disclosed. Metabolic syndrome is a combination of medical disorders that increase the risk of developing cardiovascular disease and diabetes. In some embodiments, the subject has normal plasma HDL levels and the methods treat the subject's metabolic syndrome independent of its effects on HDL levels. In some embodiments, the method involves administering to the subject an miR-33 inhibitor in a therapeutically effective amount to treat or ameliorate one or more symptoms of a metabolic syndrome.

In some embodiments, the subject has insulin resistance or non-alcoholic hepatic steatosis (fatty liver), atherosclerosis, or a combination thereof, and the method improves these conditions.

Compositions and methods for treating and preventing atherosclerosis are disclosed. In some embodiments, the subject is at risk of atherosclerosis or atherosclerotic plaque rupture. Also disclosed are compositions and methods for increasing cholesterol efflux from peripheral cells. In certain embodiments, the peripheral cells are cholesterol-laden macrophages present in atherosclerotic plaques.

Endogenous microRNAs can be silenced using antagomirs, which are small RNA that are complementary to the microRNAs target. The miR-33 inhibitor is preferably an antagomir having a single-stranded nucleic acid sequence that is complementary to at least 12 contiguous nucleotides in miR-33, wherein the antisense oligonucleotide forms a duplex with miR-33 under physiological conditions. The antisense oligonucleotide preferably comprises one or more nucleotide modifications that increase stability of the antisense oligonucleotide in the presence of a nuclease.

### BRIEF DESCRIPTION OF THE DRAWINGS

FIG. 1A is a schematic representation of the Srebf2 gene locus demonstrating the miR-33 coding sequence within intron 16 and its conservation among species (reference=mouse genome). FIG. 1B is a bar graph showing relative expression of miR-33 (first set of bars), Srebf2 (second set of bars), Srebf1 (third set of bars) and Abca1 (fourth set of bars) in control macrophages (left bars) or macrophages loaded with cholesterol by AcLDL treatment (right bars) or depleted of cholesterol by statin treatment (middle bars). FIG. 1C is a bar graph showing relative miR-33 expression in liver of C57BL6 mice (n=5 per group) fed a chow diet (left bar), high-fat diet (HFD, middle bar) or rosuvastatin-supplemented diet (statin, right bar). FIG. 1D is a bar graph showing relative miR-33 expression in liver (first set of bars) and peritoneal macrophages (PMø, second set of bars)) from Ldir^{-/-} mice fed a chow (left bars) or HFD (right bars) for 12 weeks (n=6 per group). FIG. 1E is a bar graph showing relative miR-33 tissue expression in the lung (first bar), liver (second bar), kidney (third bar), brain (fourth bar), spleen (fifth bar), heart (sixth bar), muscle (seventh bar), and aorta (eighth bar) of C57BL6 mice (n=3). Data are the mean±s.e.m and are representative of ≧3 experiments. *p≦0.05, **p≦0.005.

FIG. 2A is a graph showing the activity of luciferase reporter constructs fused to the 3′UTR of human ABCA1 (triangle), human NPC1 (square), mouse ABCG1 (open circle) or human ABCG1 (closed circle) in HEK293 cells transfected with increasing concentrations (0, 5, 50 or 500 ng) of control miR or miR-33. FIGS. 2B-2D are bar graphs showing luciferase reporter activity of the hABCA1 (FIG. 2B), hNPC1 (FIG. 2C) and mABCG1 (FIG. 2D) luciferase constructs in COS-7 cells transfected with control miR (closed bars) or miR-33 (open bars) for normal 3′UTRs target sites (left set of bars) or 3′UTRs target sites containing point mutations (“PM”, second and third set of bars). *p≦0.05.

FIGS. 3A and 3B are bar graphs showing cholesterol efflux to apoA1 (FIG. 3A) and HDL (FIG. 3B) in mouse J774 (first three sets of bars) and human THP-1 macrophages (last three sets of bars) unstimulated (first and fourth set of bars), stimulated with AcLDL (second and fifth set of bars), or stimulated with T0901317 (third and sixth set of bars) and expressing a control miR (closed bars) or miR-33 (open bars). FIG. 3C is a bar graph showing cholesterol efflux to apoA1 in mouse J774 (first three sets of bars) and human THP-1 macrophages (last three sets of bars) unstimulated (first and fourth set of bars), stimulated with AcLDL (second and fifth set of bars) or stimulated with T0901317 (third and sixth set of bars) and expressing a control inhibitor (closed bars) or anti-miR-33 (open bars). FIG. 3D is a graph showing plasma HDL levels as a function of time (days) in mice infected with the control (square), miR-33 (circle) or anti-miR-33 (triangle) lentiviruses. (n=6). FIG. 3E is a bar graph showing percentage change in plasma HDL 6 days following lentiviral delivery of miR33 (open bars) or anti-miR-33 (closed bars) (relative to control). Data are the mean±s.e.m. *p≦0.05.

FIG. 4A is a bar graph showing relative expression of miR-33 (first set of bars), miR-611 (second set of bars), and miR-515-3p (third set of bars) in cholesterol loaded (closed bars) and cholesterol depleted (open bars) macrophages. FIG. 4B is a graph showing relative expression of miR-33 (circles) and SREBF2 (squares) in human THP-1 macrophages treated with simvastatin as a function of time of treatment (hours). FIG. 4C contains two bar graphs showing total hepatic cholesterol levels (left graph, μg/mg protein) and plasma cholesterol levels (right graph, mg/dl) of C57BL6 mice (n=5 per group) fed a chow (left bars), high-fat (HFD, middle bars) or rosuvastatin-supplemented diet (statin, right bars). FIG. 4D is a bar graph showing relative gene expression of Abca1 (first set of bars), Hmgcr (second set of bars) and Srebf2 (third set of bars) in liver from mice fed a chow (left bars), high-fat (HFD, middle bars) or rosuvastatin-supplemented (statin, right bars) diet. Hmgcr mRNA expression was monitored as a cholesterol-responsive gene. Data are the mean±s.e.m and are representative of 3 or more experiments. *p≦0.05.

FIG. 5A is a bar graph showing relative expression of miR-33 (first set of bars) and Abca1 (second set of bars) in peritoneal macrophages from 6 month old C57BL6 (closed bars) or Apoe^{-/-} (open bars) mice. FIG. 5B is a bar graph showing total cholesterol (bottom set of bars) and cholesterol ester (CE, top set of bars) content of peritoneal macrophages from 6 month old C57BL6 (closed bars) or Apoe^{-/-} (open bars) mice. FIG. 5C is a bar graph showing relative miR-33 expression in macrophage [J774 (first bar), RAW (second bar), THP1 (third bar)], hepatic [HEPA (fourth bar), HEPG2 (fifth bar)], or endothelial [HUVEC (sixth bar), Eahy (seventh bar)] cell lines. Data are the mean±s.e.m and are representative of 3 or more experiments. *p<0.05.

FIG. 6A is a schematic representation of three putative miR-33 binding sites in mouse and human ABCA1. FIG. 6B is a schematic representation of two putative miR-33 binding sites in mouse ABCG1. FIG. 6C is a schematic representation of one putative miR-33 binding sites in mouse NPC1 and two putative miR-33 binding sites in human NPC1. (Mmu=mouse). The sequence for mmu-miR-33 (SEQ ID NO:23) is shown. Also shown are sequences for position 134-140 of mouse ABCA1 3′UTR (SEQ ID NO:24), position 139-145 of mouse ABCA1 3′UTR (SEQ ID NO:25), position 149-155 of mouse ABCA1 3′UTR (SEQ ID NO:26), position 717-723 of mouse ABCG1 3′UTR (SEQ ID NO:27), position 727-733 of mouse ABCG1 3′UTR (SEQ ID NO:28), position 327-333 of mouse NPC1 3′UTR (SEQ ID NO:29), position 321-327 of human NPC1 3′UTR (SEQ ID NO:30), and position 443-449 of human NPC1 3′UTR (SEQ ID NO:31).

FIG. 7A-7C are bar graphs showing mRNA levels of ABCA1 (FIG. 7A), ABCG1 (FIG. 7B) and NPC1 (FIG. 7C) in primary mouse macrophages transfected with control miR (first three bars) or miR-33 (last three bars) unstimulated (first and fourth bars), upon stimulation with AcLDL (Ac, second and fifth bars), or upon stimulation with T0901317 (T, third and sixth bars). FIG. 7D is a bar graph showing 3′UTR activity of hNPC (first two bars, with 2 putative miR-33 binding sites) and mNPC1 (last two bars, with 1 putative miR-33 binding site) in the presence of a control miR (closed bars) or miR-33 (open bars). *p<0.05.

FIGS. 8A-8C contain bar graphs showing relative protein levels of ABCA1 (FIG. 8A), ABCG1 (FIG. 8B) and NPC1 (FIG. 8C) in mouse peritoneal macrophages transfected with control miR (first set of bars), miR-33 (second set of bars), control inhibitor (third set of bars), or anti-miR-33 (fourth set of bars) that were unstimulated (left bars), stimulated with AcLDL (middle bars), or stimulated with T0901317 (right bars). FIGS. 8D-8F contain bar graphs showing relative protein levels of ABCA1 (FIG. 8D), ABCG1 (FIG. 8E) and NPC1 (FIG. 8F) in mouse peritoneal macrophages transfected with increasing concentrations of control miR (second through sixth bars, nM), increasing concentrations of miR-33 (seventh through twelfth bars, nM) that were unstimulated (open bars) or stimulated with T0901317 (closed bars). FIG. 8G-8I contains three bar graphs showing relative protein levels of ABCA1 (FIG. 8G), ABCG1 (FIG. 8H) and NPC1 (FIG. 8I) in mouse peritoneal macrophages transfected with combinations of control miR, miR-33, control inhibitor, and anti-miR-33 that were unstimulated (closed bars) or stimulated with T0901317 (open bars). *p<0.05.

FIGS. 9A to 9L are bar graphs showing relative protein levels of ABCA1 (FIG. 9A, 9D, 9G, 9J), ABCG1 (FIG. 9B, 9E, 9H, 9K) and NPC1 (FIG. 9C, 9F, 9I, 9L) in human THP-1 (FIG. 9A, 9B, 9C), mouse peritoneal macrophages (FIG. 9D, 9E, 9F), human HepG2 (FIG. 9G, 9H, 9I), and mouse HEPA (FIG. 9J, 9K, 9L) transfected with a control inhibitor (first set of bars) and anti-miR-33 (right set of bars) that were unstimulated (left bars) or stimulated with AcLDL (middle bars) or T0901317 (right bars).

FIG. 10A is a bar graph showing percent cholesterol efflux to ApoA1 in HEPG2 (left set of bars) or Fu5aH (right set of bars) hepatic cells expressing a control miR (closed bar) or miR-33 (open bar). FIG. 10B is a bar graph showing percent cholesterol efflux to ApoA1 in THP-1 (left set of bars) or HEPG2 (right set of bars) cells expressing a control inhibitor (closed bar) or anti-miR-33 (open bar). Data are the mean±s.e.m and are representative of 3 experiments. *p≦0.05.

FIGS. 11A and 11B are bar graphs showing relative expression of hepatic ABCA1 (first set of bars), ABCG1 (second set of bars) and NPC1 (third set of bars) expression from mice (n=6) 6 days after infection with control (closed bars), miR-33 (FIG. 11A, open bars) or anti-miR-33 (FIG. 11B, open bars) lentiviruses.

FIGS. 12A and 12B are bar graphs showing mRNA expression (fold change) of (from left to right) ACC1, SREBP2, MB OAT2, IRS2, CROT, SCD, HADHB, SREBP1C, FAS, CPT1a, and AMPKα genes in Huh7 cells overexpressing miR-33b (FIG. 12A) or treated with anti-miR-33b (FIG. 12B). FIGS. 12C and 12D are bar graphs showing protein expression (arbitrary units) of (from left to right) IRS2, AMPKα CROT, CPT1a, and HADHB, in Huh7 cells overexpressing miR-33b (FIG. 12C, open bar), overexpressing control miR (FIG. 12C, solid bar), treated with anti-miR-33b (FIG. 12D, open bar), or treated with control inhibitor (FIG. 12D, solid bar).

FIGS. 13A and 13B are graphs showing relative rate of β-oxidation in Huh7 cells transfected with control (open bars), miR-33 (FIG. 13A), and anti-miR-33b (FIG. 13B). FIG. 13C is a bar graph showing triglyceride (TG) content (μg TG/mg protein) of Huh7 cells transfected with control (left set of bars) or miR-33b (right set of bars) at 0 h (open bars) and 24 h (solid bars) of starvation. FIG. 13D is a bar graph showing triglyceride synthesis (cpm/mg protein) of Huh7 cells transfected with control (left set of bars) or miR-33b (right set of bars) and stimulated (solid bars) or not stimulated (open bars) with insulin. FIG. 13E is a bar graph showing triglyceride content (μg TG/mg protein) of transgenic Drosophila overexpressing miR-33 (right set of bars) or control (left set of bars) Cg-DsRed transgene in the fat body before (open bars) and 24 hours after (solid bars) starvation.

FIGS. 14A and 14B are bar graphs showing AKT phosphorylation (p-AKTSer473/total-AKT (arbitrary units)) in hepatic Huh7 cells transfected with control miR (FIGS. 14A and 14B, bars 1-3), miR-33b (FIG. 14A, bars 4-6), or miR-33b and IRS2 (FIG. 14B, bars 4-6), and treated with insulin for 0, 5, and 15 minutes. FIG. 14C is a bar graph showing 2-deoxyglucose (2-DOG) uptake (% of control) in Huh7 cells transfected with control miR (bars 1-2) or miR-33b (bars 3-4) and treated with insulin (solid bar) or untreated (open bar). FIG. 14D is a bar graph showing Luciferase activity in HEK293 cells after cotransfection of miR-33b (open bars) or control miR (solid bars) with the 3′ UTR of human Sirt6 (first set of bars) or a 3′ UTR of human Sirt6 having a point mutation (PM) in the miR-33 target sites. Results are expressed as the percentage of 3′ UTR activity of control miR. FIG. 14E is a bar graph showing SIRT6 mRNA (fold change) from HepG2 after overexpressing (sold bar) or inhibiting (open bar) endogenous miR-33b levels. FIG. 14F is a bar graph showing relative rate of cellular β-oxidation (14CO₂ released from [¹⁴C]-Oleate oxidation) after SIRT6 knockdown using siRNA SIRT6 (sold bar) or nonsense control (open bar). Data are the mean±SEM and are representative of at least three experiments. *P≦0.05.

FIG. 15A is a bar graph showing relative miR-33 expression in livers of anti-miR33 treated mice (solid bar) compared to mice treated with control anti-miR (open bar). FIG. 15B is a bar graph showing mRNA expression of ABCA1, ABCG1, HADHB, CROT, CPT1a, INSIG, HMGCR, and SREBP2 following anti-miR33 treatment (right bars) compared to no treatment (left bars), or control anti-miR (middle bars). FIG. 15C is a bar graph showing ABCA1 (left set of bars) and ABCG1 (right set of bars) protein expression (relative density) in the liver of mice treated with anti-miR33 (solid bars) compared to controls (open bars). *p<0.05.

FIG. 16A is a bar graph showing total cholesterol (mg/dL) from mice treated for 4 weeks with PBS (no treatment, first bar), control anti-miR (second bar) and anti-miR33 (third bar). FIG. 16B is a bar graph showing HDL levels (mg/dl) after 4 weeks with PBS (no treatment, first bar), control anti-miR (second bar) and anti-miR33 (third bar). FIG. 16C is a graph showing FPLC profiles of control anti-miR (open circles) and anti-miR33 (closed circles) treated mice. *p<0.05.

FIG. 17A-17C are bar graphs showing reverse cholesterol transport (RCT, % injected cpm) to serum (FIG. 17A), liver (FIG. 17B), and feces (FIG. 17C) measured at 6 h (first set of bars), 24 h (second set of bars) and 48 h (third set of bars) after ³H-cholesterol labeled macrophage injection in anti-miR33 treated mice (solid bars) compared to controls (open bars). *p<0.05.

FIG. 18 is a bar graph showing lesion area (mm²) from mice at baseline (first bar, after 14 w Western diet) and after 4 weeks with PBS (second bar), control anti-miR (third bar), or anti-miR33 (fourth bar) treatment. *p<0.05.

FIG. 19A is a bar graph showing CD68-positive area (mm²) in mice after treatment with PBS (first bar), control anti-miR (second bar), and anti-miR33 (third bar) for 4 weeks. FIG. 19B is a bar graph showing collagen staining (% of lesion area) in PBS (first bar), control anti-miR (second bar), and anti-miR33 (third bar) treated mice. FIG. 19C is a bar graph showing oil red 0 staining for neutral lipids (% lesion area) in mice after treatment with PBS (first bar), control anti-miR (second bar), and anti-miR33 (third bar) for 4 weeks. *p<0.05.

FIG. 20 is a bar graph showing relative expression of Abca1 mRNA in plaque CD68+ macrophages that were isolated by laser capture microdissection from mice treated with PBS, control anti-miR, or anti-miR-33 compared to baseline, and analyzed by qRT-PCR analysis. *p≦0.05 compared to all other groups. *p<0.05.

FIG. 21 is graph showing cumulative distribution function (CDF) for mRNA of lesional macrophages isolated by laser capture in anti-miR33 (black line) treated mice compared to control anti-miR (gray line) treated mice.

### DETAILED DESCRIPTION OF THE INVENTION

### I. Definitions

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience, 1999; and Robert A. Meyers (ed.), Molecular Biology and Biotechnology, a Comprehensive Desk Reference, published by VCH Publishers, Inc., 1995; Sambrook and Russell. (2001) Molecular Cloning: A Laboratory Manual 3rd. edition, Cold Spring Harbor Laboratory Press.

To facilitate understanding of the disclosure, the following definitions are provided:

The term “metabolic syndrome” refers to a group of risk factors that contribute to an increased risk of developing cardiovascular disease and diabetes. These risk factors include central obesity, elevated triglycerides (e.g., ≧150 mg/dl), reduced HDL cholesterol (e.g., <40 mg/dL (male), <50 mg/dL (female)), elevated blood pressure (e.g., ≧130/85 mmHg), and raised fasting plasma glucose (e.g., ≧6.1 mmol/L (110 mg/dl)). However, no one risk factor is always present. For example, in some cases, subjects with metabolic syndrome have normal HDL levels and treatments to raise HDL are not effective to treat metabolic syndrome in those subjects.

The terms “reverse cholesterol transport” or “RCT” refers to the process by which accumulated cholesterol is removed from peripheral tissues back to the liver via the plasma.

The term “cholesterol efflux” refers to an RCT process where accumulated cholesterol is removed from macrophages in the subintima of the vessel wall (e.g., by high density lipoprotein or ATP-binding membrane cassette transporter A1 (ABCA1)). Impaired cholesterol efflux results in increased atherosclerosis.

The term “microRNA” or “miRNA” refers to double stranded RNA about 20 to 25 nucleotides, more preferably 21 to 23 nucleotides, in length that binds to the 3′-untranslated regions (3′-UTR) of specific mRNAs and regulates gene expression.

An “isolated” nucleic acid molecule or polynucleotide is a nucleic acid molecule that is identified and separated from at least one substance with which it is ordinarily associated in the natural source. The isolated nucleic can be, for example, free of association with all components with which it is naturally associated. An isolated nucleic acid molecule is other than in the form or setting in which it is found in nature.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The term “endogenous” with regard to a nucleic acid refers to nucleic acids normally present in the host.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “nucleic acid” may be used to refer to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The term “oligonucleotide” refers to a single-stranded nucleic acid polymer of a defined sequence that can base-pair to a second single-stranded nucleic acid polymer that contains a complementary sequence.

The term “oligoribonucleotide” refers to an oligonucleotide containing ribonucleotides.

The term “nucleotide” refers to one or more monomeric subunits of an oligonucleotide agent. The term “nucleotide” can also generally refer to a modified nucleotide or surrogate replacement moiety.

The term “ribonucleotide” is a nucleotide in which a purine or pyrimidine base is linked to a ribose molecule.

The term “duplex” or “double-stranded” refers to the linkage of two nucleic acid polymers by complementary base pairing.

The term “complementary” and “complementarity” refers to the rules of Watson and Crick base pairing. For example, A (adenine) bonds with T (thymine) or U (uracil), G (guanine) bonds with C (cytosine). For example, DNA contains an antisense strand that is complementary to its sense strand. A nucleic acid that is 95% identical to a DNA antisense strand is therefore 95% complementary to the DNA sense strand.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

The term “treat” or “treatment” as used herein means the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit,” means to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “prevent” as used herein does not require absolute forestalling of the condition or disease but can also include a reduction in the onset or severity of the disease or condition. Thus, if a therapy can treat a disease in a subject having symptoms of the disease, it can also prevent that disease in a subject who has yet to suffer some or all of the symptoms.

The term “therapeutically effective” means that the amount of the composition used is of sufficient quantity to ameliorate one or more causes or symptoms of a disease or disorder. Such amelioration only requires a reduction or alteration, not necessarily elimination.

### II. Formulations

A. miRNA Inhibitors

Cholesterol metabolism is tightly regulated at the cellular level. miRNA miR-33 is shown herein to modulate the expression of genes involved in cellular cholesterol transport. miR-33a and b are intronic miRNAs located within the genes encoding sterol-regulatory element-binding factor-2 (SREBF-2) and -1 (SREBF-1), transcriptional regulators of cholesterol and fatty acid synthesis, respectively. miR-33a and miR-33b differ by 2 of 19 nucleotides in their mature form but are identical in the seed sequence which dictates binding to the 3′UTR of genes. In mouse and human cells, miR-33 is shown to inhibit the expression of the ATP-binding cassette (ABC) transporter ABCA1, thereby attenuating cholesterol efflux to apolipoprotein A1. In mouse macrophages, miR-33 also targets ABCG1, reducing cholesterol efflux to nascent high-density lipoprotein (HDL). Lentiviral delivery of miR-33 to mice repressed ABCA1 expression in the liver, reducing circulating HDL levels. Conversely, silencing of miR-33 in vivo increases hepatic expression of ABCA1 and plasma HDL levels. Moreover, silencing of miR-33 in vivo increases cholesterol-efflux in peripheral macrophages and reduces the size of atherosclerotic plaques. Thus, miR-33 can regulate HDL biogenesis in the liver, cellular cholesterol efflux, and atherosclerotic plaque size.

MiR-33 is also shown to inhibit the expression of carnitine O-octaniltransferase (CROT), Carnitine palmitoyltransferase 1A (CPT1a) and hydroxyacyl-CoA-dehydrogenase (HADHB), reduce fatty acid oxidation in hepatic cells, and target the insulin receptor substrate 2 (IRS-2). In addition, miR-33 has been shown to inhibit the expression of AMP-activated protein kinase (PRKAA1). In some embodiments, miR-33 has these effects independent of its ability to elevating plasma high density lipoprotein (HDL) levels. Therefore miR-33 inhibitors are provided for use in treating metabolic syndrome independent of its effect on HDL plasma levels. In addition, miR-33 inhibitors decrease SREBP-1 expression, decrease genes involved in fatty acid synthesis (e.g., FAS, ACACA, ACLY), and reduce plasma VLDL triglycerides. Useful miR-33 inhibitors can be obtained by screening libraries of known compounds, including compounds of unknown function, to see if they inhibit miR-33, as described in the examples. Preferred inhibitors are compounds which are orally bioavailable, and exhibit low toxicity.

1. Antagomirs

In some embodiments, the miR-33 inhibitor is an antagomir. An “antagomir” refers to a single stranded, double stranded, partially double stranded or hairpin structured oligomer or polymer of ribonucleic acid (RNA) or deoxyribonucleic acid (DNA) or both or modifications thereof, which is antisense with respect to its miRNA target.

Examples of antagomirs and other miRNA inhibitors are described in WO2009/20771, WO2008/91703, WO2008/046911, WO2008/074328, WO2007/90073, WO2007/27775, WO2007/27894, WO2007/21896, WO2006/93526, WO2006/112872, WO2007/112753, WO2007/112754, WO2005/23986, or WO2005/13901, all of which are hereby incorporated by reference.

Custom designed Anti-miR™ molecules are commercially available from Applied Biosystems. Thus, in some embodiments, the antagomir is an Ambion® Anti-miR™ inhibitor. These molecules are chemically modified and optimized single-stranded nucleic acids designed to specifically inhibit naturally occurring mature miRNA molecules in cells. For example, product ID AM12607 from Applied Biosystems is an Ambion® Anti-miR™ inhibitor targeting human miR-33a.

Custom designed Dharmacon Meridian™ microRNA Hairpin Inhibitors are also commercially available from Thermo Scientific. These inhibitors include chemical modifications and secondary structure motifs. For example, Vermeulen et al. reports in U.S. Patent Publication 2006/0223777 the identification of secondary structural elements that enhance the potency of these molecules. Specifically, incorporation of highly structured, double-stranded flanking regions around the reverse complement core significantly increases inhibitor function and allows for multi-miRNA inhibition at subnanomolar concentrations. Other such improvements in antagomir design are contemplated for use in the disclosed methods.

In preferred embodiments, the disclosed antagomir includes a region of sufficient nucleotide length and sufficient complementarity to miR-33 that the antagomir forms a duplex with miR-33. Given the sequence of miR-33, an antagomir can be designed according to the rules of Watson and Crick base pairing.

Thus, the antagomir can be an antisense oligonucleotide having a single-stranded nucleic acid sequence that is complementary to at least 8, 9, 10, 11, 12, 13, 14, 15, 16, 17, 18, 19, 20, 21, 22, 23, or 24 contiguous nucleotides in miR-33, wherein the antisense oligonucleotide forms a duplex with miR-33 under physiological conditions.

The following is a schematic of human miR-33a stem loop (SEQ ID NO:43):

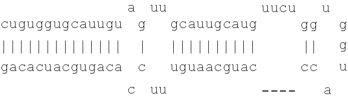

In certain embodiments, human miR-33a can have the nucleic acid sequence GUGCAUUGUAGUUGCAUUGCA (SEQ ID NO:32). Thus, in certain embodiments, the single-stranded nucleic acid sequence hybridizes under stringent conditions to an oligonucleotide having the sequence of SEQ ID NO: 32. In certain embodiments, human miR-33a* can have the nucleic acid sequence: CAAUGUUUCCACAGUGCAUCAC (SEQ ID NO:33). Therefore, in certain embodiments, the single-stranded nucleic acid sequence hybridizes under stringent conditions to an oligonucleotide having the of nucleic acid sequence SEQ ID NO:33.

The following is a schematic of human miR-33b stem loop (SEQ ID NO:44):

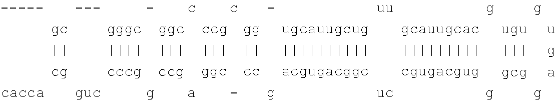

In certain embodiments, human miR-33b can have the nucleic acid sequence of GUGCAUUGCUGUUGCAUUGC (SEQ ID NO:34). Thus, in certain embodiments, the single-stranded nucleic acid sequence hybridizes under stringent conditions to an oligonucleotide having the sequence of SEQ ID NO: 34. In certain embodiments, human miR-33b* can have the nucleic acid sequence CAGUGCCUCGGCAGUGCAGCCC (SEQ ID NO:35). Therefore, in certain embodiments, the single-stranded nucleic acid sequence hybridizes under stringent conditions to an oligonucleotide having the of nucleic acid sequence SEQ ID NO:35.

In some embodiments, a single-stranded nucleic acid sequence hybridizes under stringent conditions to an oligonucleotide having the sequence of SEQ ID NO:32, and hybridizes under stringent conditions to an oligonucleotide having the sequence of SEQ ID NO:34. In some embodiments, a single-stranded oligonucleotide has a nucleobase sequence that is complementary to at least twelve contiguous nucleotides of SEQ ID NO: 32 and/or SEQ ID NO:34. In some embodiments, a single-stranded oligonucleotide has a nucleobase sequence that is complementary to at least seven contiguous nucleotides of SEQ ID NO:32 and/or SEQ ID NO:34. In some embodiments, a single-stranded oligonucleotide comprises the sequence 5′-CAATGCANNN-3′ (SEQ ID NO:36) or 5′-CAAUGCANNN-3′ (SEQ ID NO:37). In some embodiments, a single-stranded oligonucleotide comprises a sequence selected from 5′-CAATGCANNNNCAATGCA-3′ (SEQ ID NO:38), 5′-CAAUGCANNNNCAAUGCA-3′ (SEQ ID NO:39), 5′-CAAUGCANNNNCAATGCA-3′ (SEQ ID NO:40), and 5′-CAATGCANNNNCAAUGCA-3′ (SEQ ID NO:41), wherein each N is independently selected from A, C, G, T, and U. In some embodiments, a single-stranded oligonucleotide has between 7 and 30, between 7 and 25, between 7 and 24, between 7 and 23, between 7 and 22, between 7 and 21, between 7 and 20, between 7 and 19, or between 7 and 18 nucleotides. In some embodiments, a single-stranded oligonucleotide has between 12 and 25, between 15 and 25, or between 17 and 25 nucleotides. In some embodiments, the single-stranded oligonucleotide is an antisense oligonucleotide. In some embodiments, the antisense oligonucleotide is a miR-33 inhibitor. In some embodiments, the single-stranded oligonucleotide consists of the sequence 5′-TGCAATGCAACTACAATGCAC-3′ (SEQ ID NO:42).

In some embodiments, miR-33 is miR-33a. In some embodiments, miR-33 is miR-33b.

In preferred embodiments, the antisense oligonucleotide contains one or more nucleotide modifications that increase stability of the antisense oligonucleotide in the presence of a nuclease. For example, one or more of the nucleotide units of the antisense oligonucleotide can be locked nucleic acid (LNA) units. In some embodiments, one or more of the nucleotide units of the antisense oligonucleotide are 2′ substituted nucleotide analogues. Additionally, one or more of the internucleoside linkages between the nucleotide units of the antisense oligonucleotide can be phosphorothioate internucleoside linkages. It is understood that the antisense oligonucleotide can include one or more different types of modifications. Thus, the antisense oligonucleotide can have LNA units, 2′ substituted nucleotide analogues, and phosphorothioate internucleoside linkages. Other modifications that are suitable for improving therapeutic use of a nucleic acid, such as an RNA molecule, can also be used with the disclosed antisense oligonucleotide.

1. Length

The antagomir can include an antisense oligonucleotide having a length of at least 8 contiguous nucleotides. Therefore, the antisense oligonucleotide can have 8, 9, 10, 11, 12, 13, 14, 15, 16, 17, 18, 19, 20, 21, 22, 23, or 24 contiguous nucleotides. The oligonucleotide is preferably less than 30 contiguous nucleotides in length. The oligonucleotide can be less than 25, 26, 27, 28, 29, 30, 31, 32, 33, 34, 35, 36, 37, 38, 39, 40 contiguous nucleotides in length

2. Complementarity

The disclosed antagomir can include an antisense oligonucleotide having a region that is at least partially, and in some embodiments fully, complementary to miR-33. It is not necessary that there be perfect complementarity between the antagomir and the target, but the correspondence must be sufficient to enable the antisense oligonucleotide to duplex with miR-33 and subsequently reduce its activity. For example, in preferred embodiments, the antisense oligonucleotide inhibits miR-33 binding to the 3′UTR of ABCA1.

The disclosed antagomir can include an antisense oligonucleotide having a region that is at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to miR-33.

Preferably, the disclosed antagomir has at least 6, 7, 8, 9, 10, 11, 12, 13, 14, 15, 16, 17, 18, 19, 20 or more contiguous nucleotides complementary to an miR-33 nucleotide sequence. In one embodiment, the disclosed antagomir has a nucleotide sequence that is complementary to miR-33. Thus, in one embodiment, the disclosed antagomir has at least 6, 7, 8, 9, 10, 11, 12, 13, 14, 15, 16, 17, 18, 19, 20 or more contiguous nucleotides that are complementary to miR-33.

In some embodiments, there will be nucleotide mismatches in the region of complementarity. In a preferred embodiment, the region of complementarity will have no more than 1, 2, 3, 4, or 5 mismatches.

In some embodiments, the antagomir is “exactly complementary” to miR-33. Thus, in one embodiment, the antagomir can anneal to miR-33 to form a hybrid made exclusively of Watson-Crick base pairs in the region of exact complementarity. Thus, in some embodiments, the antagomir specifically discriminates a single-nucleotide difference. In this case, the antagomir only inhibits miR-33 activity if exact complementarity is found in the region of the single-nucleotide difference.

3. Modifications

The disclosed antagomirs include oligomers or polymers of ribonucleic acid (RNA) or deoxyribonucleic acid (DNA) or both or modifications thereof. This term includes oligonucleotides composed of naturally occurring nucleobases, sugars, and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target, and/or increased stability in the presence of nucleases.

The antagomir oligonucleotide can include unmodified RNA and DNA as well as RNA and DNA that have been modified, e.g., to improve efficacy, and polymers of nucleoside surrogates. “Unmodified” RNA refers to a molecule in which the components of the nucleic acid, namely sugars, bases, and phosphate moieties, are the same or essentially the same as that which occur in nature, preferably as occur naturally in the human body. “Modified” RNA, as used herein, refers to a molecule where one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs.

The disclosed antagomir oligonucleotide can be modified to enhance resistance to nucleases. The antagomir oligonucleotide can include nucleotide modification that stabilized it against nucleolytic degradation. The oligomer can be a totalmer, mixmer, gapmer, tailmer, headmer or blockmer. A “totalmer” is a single stranded oligonucleotide that only comprises non-naturally occurring nucleotides.

The term “gapmer” refers to an oligonucleotide composed of modified nucleic acid segments flanking at least 5 naturally occurring nucleotides (i.e., unmodified nucleic acids).

The term “blockmer” refers to a central modified nucleic acid segment flanked by nucleic acid segments of at least 5 naturally occurring nucleotides.

The term “tailmer” refers to an oligonucleotide having at least 5 naturally occurring nucleotides at the 5′-end followed by a modified nucleic acid segment at the 3′-end.

The term “headmer” refers to oligonucleotide having a modified nucleic acid segment at the 5′-end followed by at least 5 naturally occurring nucleotides at the 3′-end.

The term “mixmer” refers to oligonucleotide which comprise both naturally and non-naturally occurring nucleotides. However, unlike gapmers, tailmers, headmers and blockmers, there is no contiguous sequence of more than 5 naturally occurring nucleotides, such as DNA units.

Modified nucleic acids and nucleotide surrogates can include one or more of: (i) replacement of one or both of the non-linking phosphate oxygens and/or of one or more of the linking phosphate oxygens; (ii) replacement of a constituent of the ribose sugar, e.g., of the 2′ hydroxyl on the ribose sugar, or wholesale replacement of the ribose sugar with a structure other than ribose; (iii) wholesale replacement of the phosphate moiety with “dephospho” linkers; (iv) modification or replacement of a naturally occurring base; (v) replacement or modification of the ribose-phosphate backbone; or (vi) modification of the 3′ end or 5′ end of the RNA, e.g., removal, modification or replacement of a terminal phosphate group or conjugation of a moiety, such as a fluorescently labeled moiety, to either the 3′ or 5′ end of RNA.

The phosphate group in a nucleic acid can be modified by replacing one of the oxygen atoms with a different substituent. One result of this modification to RNA phosphate backbones can be increased resistance of the oligoribonucleotide to nucleolytic breakdown. Thus, it can be desirable in some embodiments to introduce alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur.

The phosphate linker can also be modified by replacement of a linking oxygen with nitrogen (bridged phosphoroamidates), sulfur (bridged phosphorothioates) and carbon (bridged methylenephosphonates). The replacement can occur at a terminal oxygen.

The phosphate group can be replaced by non-phosphorus containing connectors. Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

A modified RNA can include modification of all or some of the sugar groups of the ribonucleic acid. For example, the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, O-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino). Oligonucleotides containing only the methoxyethyl group (MOE) (OCH₂CH₂OCH₃, a PEG derivative) exhibit nuclease stabilities comparable to those modified with the robust phosphorothioate modification.

“Deoxy” modifications include hydrogen (i.e., deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

Thus, the antagomir can include a 2′-deoxy, 2′-deoxy-2′-fluoro, 2′-β-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, the antagomir includes at least one 2′-O-methyl-modified nucleotide, and in some embodiments, all of the nucleotides of the antagomir include a 2′-O-methyl modification.

The sugar group can also contain one or more carbons that possess the opposite stereochemical configuration than that of the corresponding carbon in ribose. Thus, a modified RNA can include nucleotides containing e.g., arabinose, as the sugar.

Modified RNAs can also include “abasic” sugars, which lack a nucleobase at C-1′. These abasic sugars can also further contain modifications at one or more of the constituent sugar atoms. The modification can also entail the wholesale replacement of a ribose structure with another entity (an SRMS) at one or more sites in the oligonucleotide agent.

The 3′ and 5′ ends of an oligonucleotide can be modified. Such modifications can be at the 3′ end, 5′ end, or both ends of the molecule. They can include modification or replacement of an entire terminal phosphate or of one or more of the atoms of the phosphate group. For example, the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The terminal atom of the spacer can connect to or replace the linking atom of the phosphate group or the C-3′ or C-5′ 0, N, S or C group of the sugar. Alternatively, the spacer can connect to or replace the terminal atom of a nucleotide surrogate (e.g., PNAs). These spacers or linkers can include e.g., —(CH₂)—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents.

Other examples of terminal modifications include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications include the addition of a methylphosphonate at the 3′-most terminal linkage; a 3′ C5-aminoalkyl-dT; 3′ cationic group; or another 3′ conjugate to inhibit 3′-5′ exonucleolytic degradation.

Terminal modifications useful for modulating activity include modification of the 5′ end with phosphate or phosphate analogs. For example, in some embodiments, oligonucleotide agents are 5′ phosphorylated or include a phosphoryl analog at the 5′ terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)₂(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl-methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-).

Adenine, guanine, cytosine and uracil are the most common bases found in RNA. These bases can be modified or replaced to provide RNAs having improved properties. For example, nuclease resistant oligonucleotides (i.e., oligoribonucleotides) can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases” and “universal bases”, can be employed. Examples include 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N.sup.4-acetyl cytosine, 2-thiocytosine, N6-methyladenine, N6-isopentyladenine, 2-methylthio-N-6-isopentenyladenine, N-methylguanines, or O-alkylated bases.

The antagomir can include an internucleotide linkage (e.g., the chiral phosphorothioate linkage) useful for increasing nuclease resistance.

Phosphorothioates (or S-oligos) are a variant of normal DNA or RNA in which one of the nonbridging oxygens is replaced by a sulfur. The sulfurization of the internucleotide bond dramatically reduces the action of endo- and exonucleases including 5′ to 3′ and 3′ to 5′ DNA POL 1 exonuclease, nucleases S1 and P1, RNases, plasma nucleases and snake venom phosphodiesterase. In addition, the potential for crossing the lipid bilayer increases. Because of these important improvements, phosphorothioates have found increasing application in cell regulation.

Phosphorothioates are made by two principal routes: by the action of a solution of elemental sulfur in carbon disulfide on a hydrogen phosphonate, or by the more recent method of sulfurizing phosphite triesters with either tetraethylthiuram disulfide (TETD) or 3H-1,2-bensodithiol-3-one 1,1-dioxide (BDTD).

One way to increase resistance is to identify cleavage sites and modify such sites to inhibit cleavage. For example, the dinucleotides 5′-UA-3′,5′-UG-3′, 5′-CA-3′,5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. Thus, the antagomir can include at least 2, 3, 4 or 5 of such dinucleotides. In certain embodiments, all the pyrimidines of an antagomir carry a 2′-modification, and the antagomir therefore has enhanced resistance to endonucleases.

An antagomir can have secondary structure, but it is preferably substantially single-stranded under physiological conditions at least in the region of the antagomir that is complementary to the miRNA. An antagomir that is substantially single-stranded is single-stranded to the extent that less than about 50% (e.g., less than about 40%, 30%, 20%, 10%, or 5%) of the antagomir is duplexed with itself. Thus, the antagomir preferably does not form hairpin loops, bulges or internal loops within the complementary region under physiological conditions.

In a preferred embodiment, the antagomir does not include a sense strand. In some embodiments, the antagomir is partially double-stranded but is single-stranded at least in the region of the antagomir that is complementary to the miRNA. The term “partially double-stranded” refers to double stranded structures wherein one strand contains fewer nucleotides than its complementary strand. In general, such partial double stranded agents will have less than 75% double stranded structure, preferably less than 50%, and more preferably less than 25%, 20% or 15% double stranded structure.

In a preferred embodiment, the antagomir is suitable for delivery to a cell in vivo, e.g., to a cell in an organism. In another embodiment, the antagomir is suitable for delivery to a cell in vitro, e.g., to a cell in a cell line in culture or a suspension. The antagomir can include a ligand that is selected to improve stability, distribution or cellular uptake of the agent. For example, the ligand can be a lipophilic moiety, e.g., cholesterol, which enhances entry of the antagomir into a cell.

The antagomir can also be encapsulated by cationic lipid particles. Cationic lipid saturation influences intracellular delivery of encapsulated nucleic acids. Cationic lipids include 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA) and 1,2-dilinolenyloxy-N,N-dimethyl-3-aminopropane (DLenDMA).

In some embodiments, the disclosed antagomir can include an aminoglycoside ligand, which can cause the antagomir to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. In some embodiments the guanidine analog (the guanidinoglycoside) of an aminoglycoside ligand is tethered to an oligonucleotide agent. In a guanidinoglycoside, the amine group on the amino acid is exchanged for a guanidine group. Attachment of a guanidine analog can enhance cell permeability of an oligonucleotide agent.

The disclosed antagomir can be expressed within cells from an expression vector having a nucleic acid encoding the antagomir. The nucleic acid sequence can be operably linked to an expression control sequence, such as a promoter. Those skilled in the art realize that any nucleic acid can be expressed in eukaryotic cells from the appropriate DNA/RNA vector.

Thus, the disclosed antagomir can be expressed from transcription units inserted into DNA or RNA vectors. The recombinant vectors can be DNA plasmids or viral vectors. Oligonucleotide agent-expressing viral vectors can be constructed based on, but not limited to, adeno-associated virus, retrovirus, lentivirus, adenovirus, or alphavirus. The recombinant vectors capable of expressing the oligonucleotide agents can be delivered as described above, and can persist in target cells. Alternatively, viral vectors can be used that provide for transient expression of nucleic acid molecules. Such vectors can be repeatedly administered as necessary. Once expressed, the disclosed antagomir interacts with miR-333 and inhibits its activity. In preferred embodiments, the at least part of the antagomir forms a duplex with endogenous miR-33, which prevents the endogenous miR-33 from binding to its target mRNA (e.g., ABCA1), which results in increased translation of the target mRNA. Delivery of oligonucleotide agent-expressing vectors can be systemic, such as by intravenous or intra-muscular administration, by administration to target cells ex-planted from a subject followed by reintroduction into the subject, or by any other means that would allow for introduction into the desired target cell (for a review see Couture et al., Trends in Genetics 12:510, 1996).

2. Small Molecule miR-33 Inhibitors

The miR-33 inhibitor can also be a small molecule inhibitor. As used herein, the term “small molecule” refers to small organic compounds, inorganic compounds, or any combination thereof that inhibits or reduces miR-33 activity; this term may include monomers or primary metabolites, secondary metabolites, a biological amine, a steroid, or synthetic or natural, non-peptide biological molecule(s).

For example, Huang and his colleagues developed a method to identify inhibitors of miRNA pathways in live human cells (Angew Chem Int Ed Engl. 2008; 47(39):7482-4). Specifically, they designed a screening assay to look for small molecules or compounds that selectively repress miRNA. They selected miR-21 as the target agent due to its documented role in preventing cell death—thereby allowing the unchecked cell proliferation associated with cancer—and its elevated levels in various cancers. Their assay contained the DNA binding sequence complementary to the miRNA, bound to a reporter such as luciferase. Under normal conditions, the miRNA binds to the complementary sequence and inhibits the translation of the reporter, such as luciferase. Candidate agents were then be added to the sample to determine whether the candidate agent reduced miRNA inhibition of reporter expression.

Thus, a method is provided that involves providing a sample having an oligonucleotide with a DNA binding sequence complementary to miR-33 under conditions that allow the binding of miR-33 to the oligonucleotide, contacting the sample with a candidate agent, detecting the level of miR-33/oligonucleotide binding, comparing the binding level to a control, a decrease in miR-33/oligonucleotide binding compared to the control identifying an miR-33 inhibitor.

The binding of miR-33 to the oligonucleotide can be detected using routine methods. In a preferred aspect, the DNA binding sequence complementary to miR-33 is operably linked to a reporter construct, such as luciferase or GFP, wherein binding of miR-33 to the oligonucleotide inhibits reporter expression.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits miRNA. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

B. Pharmaceutical Excipients

The miR-33 inhibitor composition can be formulated for administration to a subject by combining the inhibitor with a pharmaceutically acceptable excipent. Methods for preparing pharmaceutical compositions are within the skill in the art, for example as described in Remington's Pharmaceutical Science, 18th ed., Mack Publishing Company, Easton, Pa. (1990), and The Science and Practice of Pharmacy, 2003, Gennaro et al.

In one embodiment, the formulations include anantagomir (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, or 0.3% glycine, for injection.

The pharmaceutical formulations can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (e.g., calcium DTPA, CaNaDTPA-bisamide), or, optionally, additions of calcium or sodium salts (for example, calcium chloride, calcium ascorbate, calcium gluconate or calcium lactate). Pharmaceutical compositions can be packaged for use in liquid form, or can be lyophilized.

For solid compositions, conventional non-toxic solid carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can include any of the carriers and excipients listed above and 10-95%, preferably 25%-75%, of one or more single-stranded oligonucleotide agents.

A formulated compound may assume a variety of states. In some examples, the composition is at least partially crystalline, uniformly crystalline, and/or anhydrous (e.g., less than 80, 50, 30, 20, or 10% water). In another example, the antagomir is in an aqueous phase, e.g., in a solution that includes water, this form being the preferred form for administration via inhalation.

The compound can be incorporated into a delivery vehicle, e.g., a liposome (particularly for the aqueous phase), or a particle (e.g., a microparticle). Generally, the compound is formulated in a manner that is compatible with the intended method of administration.

The MiR-33 inhibitor composition formulations can include liposomes, such as surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug.

Long-circulating liposomes enhance the pharmacokinetics and pharmacodynamics of DNA and RNA, particularly compared to conventional cationic liposomes which are known to accumulate in tissues of the MPS. Long-circulating liposomes are also likely to protect drugs from nuclease degradation to a greater extent compared to cationic liposomes, based on their ability to avoid accumulation in metabolically aggressive MPS tissues such as the liver and spleen.

The types of pharmaceutical excipients that are useful as carrier include stabilizers such as human plasma albumin (HSA), bulking agents such as carbohydrates, amino acids and polypeptides; pH adjusters or buffers; salts such as sodium chloride; and the like. These carriers may be in a crystalline or amorphous form or may be a mixture of the two. Bulking agents that are particularly valuable include compatible carbohydrates, polypeptides, amino acids or combinations thereof. Suitable carbohydrates include monosaccharides such as galactose, D-mannose, and sorbose; disaccharides, such as lactose and trehalose; cyclodextrins, such as 2-hydroxypropyl-.beta.-cyclodextrin; and polysaccharides, such as raffinose, maltodextrins, and dextrans; alditols, such as mannitol and xylitol. A preferred group of carbohydrates includes lactose, threhalose, raffinose maltodextrins, and mannitol. Suitable polypeptides include aspartame. Amino acids include alanine and glycine, with glycine being preferred.

C. Additional Active Agents

A compound can be formulated in combination with another agent. In some embodiments, the compound is formulated with another therapeutic agent. In some embodiments, the compound is formulated with an agent that stabilizes the oligonucleotide agent, e.g., a protein that complexes with the oligonucleotide agent. Still other agents include chelators, e.g., EDTA (e.g., to remove divalent cations such as Mg²⁺), salts, and RNAse inhibitors (e.g., a broad specificity RNAse inhibitor such as RNAsin).

In one embodiment, the antagomir preparation includes another antagomir, e.g., a second antagomir that can down-regulate expression of a second miRNA. In some embodiments, the agents are directed to the same target nucleic acid but different target sequences. In another embodiment, each antagomir is directed to a different target.

A compound can be formulated in combination with one or more other compounds, especially other compounds involved in inhibition of cholesterol synthesis or uptake, such as a statin, bile acid sequestrants, cholesterol absorption inhibitors such as fibrate, nicotinic acid, etc.

### III. Methods of Making Antagomirs and Formulations Thereof

An antagomir, such as a single-stranded oligonucleotide agent, can be constructed using chemical synthesis and/or enzymatic ligation reactions using procedures known in the art. For example, an antagomir can be chemically synthesized using naturally occurring nucleotides or variously modified nucleotides designed to increase the biological stability of the molecules or to increase the physical stability of the duplex formed between the antagomir and target nucleic acids, e.g., phosphorothioate derivatives and acridine substituted nucleotides can be used. Other appropriate nucleic acid modifications are described herein. Alternatively, the antagomir can be produced biologically using an expression vector into which a nucleic acid has been subcloned in an antisense orientation (i.e., RNA transcribed from the inserted nucleic acid will be of an antisense orientation to a target nucleic acid of interest (e.g., miR-33).

### IV. Methods of Administration and Disorders to be Treated

A. Methods of Administration

A miR-33 inhibitor may be administered in a number of ways depending upon whether local or systemic treatment is desired and upon the area to be treated. Administration may be topical (including ophthalmic, intranasal, transdermal, intrapulmonary), oral or parenteral. Parenteral administration includes intravenous drip, subcutaneous, intraperitoneal or intramuscular injection, or intrathecal or intraventricular administration.

Nucleic acid molecules can be administered to cells by a variety of methods known to those of skill in the art, including, but not restricted to, encapsulation in liposomes, by ionophoresis, or by incorporation into other vehicles, such as hydrogels, biodegradable nanocapsules, and bioadhesive microspheres, or by proteinaceous vectors.

The antagomir can be administered to the subject either as a naked oligonucleotide agent, in conjunction with a delivery reagent, or as a recombinant plasmid or viral vector which expresses the oligonucleotide agent.

The miR-33 inhibitor composition can be administered to the subject by any means suitable for delivering the agent to the cells of the tissue at or near the area of unwanted miR-33 expression. For example, an MiR-33 inhibitor composition that targets miR-33 can be delivered directly to the liver, or can be conjugated to a molecule that targets the liver. Exemplary delivery methods include administration by gene gun, electroporation, or other suitable parenteral administration route.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant comprising a porous, non-porous, or gelatinous material).

The miR-33 inhibitor composition can be provided in sustained release composition. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

The miR-33 inhibitor composition can be administered in a single dose or in multiple doses. Where the administration of the antagomir is by infusion, the infusion can be a single sustained dose or can be delivered by multiple infusions.

B. Symptoms or Disorders to be Treated

1. Metabolic Syndrome

Metabolic syndrome is a combination of medical disorders that increase the risk of developing cardiovascular disease and diabetes. It affects one in five people, and prevalence increases with age. Metabolic syndrome is also known as metabolic syndrome X, syndrome X, insulin resistance syndrome, Reaven's syndrome, and CHAOS (Australia).

There are currently two major definitions for metabolic syndrome provided by the International Diabetes Federation and the revised National Cholesterol Education Program, respectively. The revised NCEP and IDF definitions of metabolic syndrome are very similar and it can be expected that they will identify many of the same individuals as having metabolic syndrome. The two differences are that IDF state that if BMI >30 kg/m², central obesity can be assumed, and waist circumference does not need to be measured. However, this potentially excludes any subject without increased waist circumference if BMI <30, whereas, in the NCEP definition, metabolic syndrome can be diagnosed based on other criteria, and the IDF uses geography-specific cut points for waist circumference, while NCEP uses only one set of cut points for waist circumference, regardless of geography. These two definitions are much closer to each other than the original NCEP and WHO definitions.

The IDF definition of the metabolic syndrome (2006) requires 1) central obesity (defined as waist circumference with ethnicity specific values) AND 2) any two of the following: a) raised triglycerides: >150 mg/dL (1.7 mmol/L), or specific treatment for this lipid abnormality; b) reduced HDL cholesterol: <40 mg/dL (1.03 mmol/L) in males, <50 mg/dL (1.29 mmol/L) in females, or specific treatment for this lipid abnormality; c) raised blood pressure: systolic BP>130 or diastolic BP>85 mm Hg, or treatment of previously diagnosed hypertension; and d) raised fasting plasma glucose: (FPG) >100 mg/dL (5.6 mmol/L), or previously diagnosed type 2 diabetes. If FPG >5.6 mmol/L or 100 mg/dL, OGTT Glucose tolerance test is strongly recommended but is not necessary to define presence of the Syndrome. Note, however, that if BMI is >30 kg/m², central obesity can be assumed and waist circumference does not need to be measured

The US National Cholesterol Education Program (NECP) Adult Treatment Panel III (2001) definition of the metabolic syndrome requires at least three of the following: 1) central obesity: waist circumference ≧102 cm or 40 inches (male), ≧88 cm or 36 inches (female); 2) dyslipidemia: TG≧1.7 mmol/L (150 mg/dl); 3) dyslipidemia: HDL-C<40 mg/dL (male), <50 mg/dL (female); 4) blood pressure ≧130/85 mmHg; 5) fasting plasma glucose ≧6.1 mmol/L (110 mg/dl).

Therefore, with either definition of metabolic syndrome, reduced HDL is not always present. Therefore, in some cases, subjects with metabolic syndrome have normal HDL levels, e.g., >40 mg/dL (1.03 mmol/L) in males, >50 mg/dL (1.29 mmol/L) in females. Consequently, it was not expected that an agent useful in elevating HDL levels could treat metabolic syndrome in all subjects, such as those with normal HDL levels. However, miR-33 inhibitors are provided to treat metabolic syndrome independent of its effect on HDL levels.

The first line treatment is change of lifestyle. However, if six months of efforts at remedying risk factors prove insufficient, then drug treatment is frequently required. Generally, the individual disorders that comprise the metabolic syndrome are treated separately. Diuretics and ACE inhibitors may be used to treat hypertension. Cholesterol drugs may be used to lower LDL cholesterol and triglyceride levels, if they are elevated, and to raise HDL levels if they are low. Use of drugs that decrease insulin resistance, e.g., metformin and thiazolidinediones, is controversial and not approved by the U.S. Food and Drug Administration.

Antagomirs are administered to individuals in need of treatment. In some embodiments, the composition increases plasma HDL levels and reduces triglyceride levels. However, the method can also treat metabolic syndrome in a subject with normal HDL levels. In some embodiments, the composition results in decreased insulin resistance. Therefore, methods of treating insulin resistance in a subject are disclosed.

In some embodiments, the treatment results in decreased non-alcoholic hepatic steatosis (fatty liver). Therefore, methods of treating non-alcoholic hepatic steatosis are disclosed.

In some embodiments, the treatment results in decreased triglyceride levels. In some embodiments, the treatment results in decreased LDL, levels VLDL levels, or a combination thereof.

The disclosed method can involve administering to the subject a pharmaceutical composition comprising a therapeutically effective amount of an miR-33 inhibitor. In some embodiments, the subject has normal plasma HDL levels. The subject may have insulin resistance. Similarly, in some embodiments, the subject has non-alcoholic hepatic steatosis (fatty liver).

In some embodiments, the method involves first detecting normal HDL levels in the subject and administering to that subject having normal HDL levels a therapeutically effective amount of an miR-33 antagomir.

The disclosed methods can also be used to increase plasma High-density lipoprotein (HDL) levels in subjects having metabolic syndrome, insulin resistance, non-alcoholic hepatic steatosis, or a combination thereof.

High concentrations of HDL have protective value against cardiovascular diseases such as ischemic stroke and myocardial infarction. Low concentrations of HDL increase the risk for atherosclerotic diseases.

The American Heart Association, NIH and NCEP provide a set of guidelines for fasting HDL levels and risk for heart disease. Low HDL cholesterol refers to levels <1.03 mmol/L (i.e., <40 mg/dL for men or <50 mg/dL for women). Medium HDL cholesterol refers to levels between 1.03 mmol/L and 1.55 mmol/L (i.e., 40-59 mg/dL). High HDL cholesterol refers to levels >1.55 mmol/L (i.e., >60 mg/dL).

Since the predominant protein in HDL is Apo A-I, chemical measurements can be used to estimate HDL concentrations present in a blood sample. However, such measurements may not indicate how well the HDL particles are functioning to reverse transport cholesterol from tissues. Since the HDL particles have a net negative charge and vary by size, electrophoresis measurements can be used to indicate the number of HDL particles and sort them by size, thus presumably function. Nuclear Magnetic Resonance fingerprinting of the particles can be used to measure both concentration and sizes. This methodology has significantly reduced costs and made such determinations more widely available clinically.

2. Atherosclerosis

Atherosclerosis is a disorder of the arteries in which fatty substances, cholesterol, cellular waste products, calcium and other substances collect in deposits along an artery wall, resulting in the formation of atherosclerotic plaques. Eventually, an atherosclerotic plaque may grow large enough that blood flow is restricted. Additionally atherosclerotic plaques may become fragile and rupture, causing blood clots to form or causing a piece of a plaque to dissociate and move through the bloodstream. Atherosclerosis can lead to serious health problems, including heart attack and stroke.

The reverse cholesterol transport pathway is a key mechanism in the prevention of atherosclerosis. Through this pathway, cholesterol is transported from peripheral cells, such as cholesterol-laden macrophages (or foam cells) present in atherosclerotic plaques, to the liver, and where the cholesterol is ultimately packaged for excretion from the body. HDL serves as a lipoprotein that accepts cholesterol from foam cells and transports the cholesterol to the liver. The cholesterol efflux from foam cells to HDL occurs in an ABCA1-dependent manner.

Evidence of an inverse association between plasma HDL cholesterol concentrations and the risk of cardiovascular disease (CVD) has led to the hypothesis that HDL protects from atherosclerosis. It has, however, been difficult to establish a causal relationship between low HDL levels and CVD risk, in part due to the fact that many different factors affect both CVD risk and HDL levels.

MiR-33 has been shown to contribute to the regulation of cholesterol homeostasis. Specifically, it has been shown that miR-33 reduces HDL levels, whereas silencing miR-33 upregulates both hepatic ABCA1 expression, and elevates plasma HDL levels, and increases cholesterol efflux (Majafi-Shoushtari S H, et al. Science, 2010, 328(5985):1566-69; Rayner R J, et al. Science, 2010, 328(5985):1570-73; Marquart T J, et al. Proc Natl Acad Sci USA, 2010 107(27)12228-32; Horie T, et al. Proc Natl Acad Sci USA, 2010 107(40)17321-26; Moore K J, et al. Trends Endocrinol. Metab. Epub Sep. 27, 2010).

However, miR-33 was not previously shown to have a direct or predictable effect on atherosclerosis. For example, a recent study attributed hyperlipidemia to miR-33 but only credited miR-21 and miR-126 for atherosclerosis (Rink C, et al. Physiol. Genomics, Epub Sep. 14, 2010).

As shown herein, inhibition of miR-33 significantly increased cholesterol efflux in macrophages. Further, in mice, inhibition of miR-33 increased reverse cholesterol transport and reduced atherosclerotic lesion area. This is surprising since it was previously shown that low HDL levels in ABCA1^{-/-} mice (virtually no HDL) does not lead to an increase in atherosclerosis.

Accordingly, provided herein are methods for increasing cholesterol efflux from peripheral cells, involving administering to the subject an inhibitor of miR-33 disclosed herein. For example, the peripheral cells can be macrophages, such as cholesterol-laden macrophages in plaques.

Also provided herein are methods for treating or preventing atherosclerosis in a subject in need thereof, involving administering to the subject an inhibitor of miR-33 disclosed herein. For example, the method can involve reducing atherosclerotic plaque size or preventing an increase in atherosclerotic plaque size in the subject. For example, the method can involve a 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, or higher decrease in plaque size.

Atherosclerosis develops from LDL becoming oxidized by free radicals, particularly reactive oxygen species (ROS). When oxidized LDL comes in contact with an artery wall, a series of reactions occur to repair the damage to the artery wall caused by oxidized LDL. The body's immune system responds to the damage to the artery wall caused by oxidized LDL by sending specialized white blood cells (macrophages and T-lymphocytes) to absorb the oxidized-LDL forming specialized foam cells. These white blood cells are not able to process the oxidized-LDL, and ultimately grow then rupture, depositing a greater amount of oxidized cholesterol into the artery wall. This triggers more white blood cells, continuing the cycle. In preferred embodiments, the disclosed method can prevent atherosclerotic plaque rupture in a subject.

These methods can further involve selecting a subject at risk for atherosclerosis. Subjects who have atherosclerosis, or are at risk for atherosclerosis, may have low levels of HDL. However, subject having or at risk for atherosclerosis may have normal levels of HDL, but may still benefit from HDL-raising therapeutic agents. Subjects who have atherosclerosis, or are at risk for atherosclerosis, may have elevated triglyceride levels. For example, in some embodiments, the methods can involve selecting a subject having a serum triglyceride level of 150 mg/dL or greater.

Atherosclerosis may be detected in subjects by angiography or stress-testing. Additional methods of detection include anatomic methods such as coronary calcium scoring by computerized tomography, carotid IMT (intimal media thickness) measurement by ultrasound, intravascular ultrasound. Physiologic methods of detection include lipoprotein subclass analysis, and measurements of HbA1c, high sensitivity C-reactive protein, and homocysteine.

3. Dosage

The disclosed pharmaceutical compositions contain miR-33 inhibitor in an effective amount to reduce atherosclerotic plaque size in a subject with atherosclerosis. In some embodiments, the miR-33 inhibitor is in an effective amount to reduce plaque size in a subject with atherosclerosis by at least 5%, 10%, 15%, 20%, 25%, 30%, 40%, or 50%.

Vessels can sustain a large increase in atherosclerotic plaque mass without luminal narrowing as a result of compensatory enlargement of the adventitial boundary. Magnetic resonance imaging (MRI) can be used to noninvasively determine arterial wall area (WA). In addition, Intravascular ultrasound (IVUS) enables accurately visualizing not only the lumen of the coronary arteries but also the atheroma (membrane/cholesterol loaded white blood cells) “hidden” within the wall.

In some embodiments, the miR-33 inhibitor is in an amount effective to prevent atherosclerosis, prevent atherosclerotic plaque rupture, or a combination thereof.

Certain factors may influence the dosage required to effectively treat a subject, including but not limited to the severity of the disease or disorder, previous treatments, the general health and/or age of the subject, and other diseases present. It will also be appreciated that the effective dosage of the antagomir used for treatment may increase or decrease over the course of a particular treatment. Changes in dosage may result and become apparent from the results of diagnostic assays. For example, the subject can be monitored after administering an antagomir composition. Based on information from the monitoring, an additional amount of the antagomir composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀s found to be effective in in vitro and in vivo animal models.

Dosage levels on the order of about 1 μg/kg to 100 mg/kg of body weight per administration are generally useful in the treatment of a disease. One skilled in the art can also readily determine an appropriate dosage regimen for administering the disclosed to a given subject. For example, the miR-33 inhibitor composition can be administered to the subject once, e.g., as a single injection. Alternatively, the miR-33 inhibitor composition can be administered once or twice daily to a subject for a period of from about three to about twenty-eight days, or from about seven to about ten days.

Thus, the miR-33 inhibitor composition can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antagomir per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antagomir per kg of bodyweight, so long as the miR-33 inhibitor is in an amount effective to increase cholesterol efflux in peripheral cells of the subject.

Delivery of an miR-33 inhibitor composition such as an antagomer directly to an organ (e.g., directly to the liver) can be at a dosage on the order of about 0.00001 mg to about 3 mg per organ, or preferably about 0.0001-0.001 mg per organ, about 0.03-3.0 mg per organ, about 0.1-3.0 mg per organ or about 0.3-3.0 mg per organ, so long as the miR-33 inhibitor is in an amount effective to increase cholesterol efflux in peripheral cells of the subject.

Where a dosage regimen comprises multiple administrations, it is understood that the effective amount of antagomir administered to the subject can include the total amount of antagomir administered over the entire dosage regimen. One skilled in the art will appreciate that the exact individual dosages may be adjusted somewhat depending on a variety of factors, including the specific antagomir being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. For instance, oral administration would require higher dosage levels than administration by intravenous injection. Variations in these dosage levels can be adjusted using standard empirical routines of optimization, which are well-known in the art. The precise therapeutically effective dosage levels and patterns are preferably determined by the attending physician in consideration of the above-identified factors.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. Because oligonucleotide agent-mediated silencing can persist for several days after administering the antagomir composition, in many instances, it is possible to administer the composition with a frequency of less than once per day, or, for some instances, only once for the entire therapeutic regimen.

In some embodiments, a subject is administered an initial dose, and one or more maintenance doses of an miR-33 inhibitor composition. The maintenance dose or doses are generally lower than the initial dose, e.g., one-half less of the initial dose. A maintenance regimen can include treating the subject with a dose or doses ranging from 0.01 μg to 75 mg/kg of body weight per day, e.g., 70, 60, 50, 40, 30, 20, 10, 5, 2, 1, 0.5, 0.1, 0.05, 0.01, 0.005, 0.001, or 0.0005 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Following successful treatment, it may be desirable to have the patient undergo maintenance therapy to prevent the recurrence of the disease state, wherein the compound of the invention is administered in maintenance doses, ranging from 0.01 μg to 100 g per kg of body weight.

In addition to treating pre-existing diseases or disorders, the miR-33 inhibitor composition can be administered prophylactically in order to prevent or slow the onset of a particular disease or disorder. In prophylactic applications, an antagomir is administered to a patient susceptible to or otherwise at risk of a particular disorder, such as disorder associated with aberrant or unwanted expression of miR-33.

The present invention will be further understood by reference to the following non-limiting examples.

### EXAMPLES

### Example 1

### Identification of miRNAs that are Differentially Regulated in Human Macrophages by Cholesterol Depletion and Cholesterol Enrichment

Materials and Methods

Materials

Chemicals were obtained from Sigma unless otherwise noted. Human lipoproteins (acetylated LDL, HDL) were obtained from Biomedical Technologies Inc (Stoughton, Mass.). The synthetic LXR ligand TO901317 is from Cayman Chemical. Human apoA1 was obtained from Meridian Life Sciences. Mouse monoclonal antibody against ABCA1 (1:1000) was purchased from Abcam. Rabbit polyclonal antibodies against ABCG1 (1:1000), SR-B1 (1:250) and NPC1 (1:1000) were obtained from Novus and mouse monoclonal HSP-90 antibody was from BD Bioscience. Polyclonal antibodies against HMGCR (1:200) and SCAP (1:200) were obtained from Santa Cruz. Secondary fluorescently-labeled antibodies were from Molecular Probes (Invitrogen).

Cell Culture

THP-1, HepG2, J774, HEPA, Fu5AH, EAhy296, COS-7 and 293T cells were obtained from American Type Tissue Collection. THP-1 and J774 cells were maintained in RPMI 1640 media (Sigma) supplemented with 10% fetal bovine plasma (FBS) and 2% penicillin-streptomycin in 10 cm² dishes at 37° C. and 5% CO². THP1 differentiation into macrophages was induced using 100 nM phorbol-12-myristate acetate (PMA) for 72 h. HepG2, HEPA, Fu5AH, COS-7 and 293T were maintained in Dulbecco's Modified Eagle Medium (DMEM) containing 10% FBS and 2% penicillin-streptomycin. EAhy296 cells were grown in DMEM containing 10% FBS and penicillin-streptomycin, L-glutamine and HAT (Sigma). Peritoneal macrophages from adult female C57BL/6J mice were harvested by peritoneal lavage four days after intraperitoneal injection of thioglycollate. The cells were maintained in culture as adherent monolayer in medium containing DMEM, 10% FBS, and 20% L-cell-conditioned medium. Cells were stimulated with 37.5 μg/ml acLDL, 10 μM TO901317, and 5 μM simvistatin at the time points as indicated in figure legends.

miRNA Microarray Analysis

Differentiated THP-1 macrophages in 6-well plates (3×10⁶ cells per well) were pre-treated overnight with 0.5% FBS in the presence or absence of simvastatin. Cells were then stimulated with 0.5% FBS media alone with either 37.5 μg/ml AcLDL or 5 μM simvastatin for 24 hours. Total RNA was extracted using Trizol (Invitrogen) and microRNA was purified from 40 μg of total RNA using the RT² qPCR-grade miRNA Isolation Kit (SABiosciences). The purity and integrity of both the total RNA sample and the enriched miRNA was verified using the Agilent Bioanalyzer (Agilent Technologies, Santa Clara, Calif.). A total of 400 ng of miRNA was reverse transcribed with the RT² miRNA First Strand kit (SABiosciences) and used for each set of Human Whole Genome miRNA Array (SABiosciences). 96-well plates were analyzed on a BioRad iCycler (BioRad Laboratories) and analysis was done using SABiosciences software. Each array was performed in triplicate from three independent experiments.

RNA Isolation and Quantitative Real-Time PCR

Total RNA was isolated using TRIzol reagent (Invitrogen) according to the manufacturer's protocol. For mRNA quantification, cDNA was synthesized using Taqman RT reagents (Applied Biosystems), following the manufacturer's protocol. Quantitative real-time PCR was performed in triplicate using iQ SYBR green Supermix (BioRad) on iCycler Real-Time Detection System (BioRad). The mRNA level was normalized to GAPDH as a house keeping gene. The primers sequences used were:

(SEQ ID NO: 1)
ABCA1, 5′-GGTTTGGAGATGGTTATACAATAGTTGT-3′
and
(SEQ ID NO: 2)
5′-CCCGGAAACGCAAGTCC-3;
(SEQ ID NO: 3)
ABCG1, 5′-TCACCCAGTTCTGCATCCTCTT-3′
and
(SEQ ID NO: 4)
5′-GCAGATGTGTCAGGACCGAGT-3′;
(SEQ ID NO: 5)
NPC1, 5′-GGTCCGCCTGTGTACTTTGT-3′
and
(SEQ ID NO: 6)
5′-GGCTTCACCCAGTCGAAATG-3′;
(SEQ ID NO: 7)
GAPDH, 5′-AACTTTGGCATTGTGGAAGG-3′
and
(SEQ ID NO: 8)
5′-ACACATTGGGGGTAGGAACA-3′;
(SEQ ID NO: 9)
SREBP2, 5′-GCGTTCTGGAGACCATGGA-3′
and
(SEQ ID NO: 10)
5′-ACAAAGTTGCTCTGAAAACAAATCA-3′;
(SEQ ID NO: 11)
SREBP1, 5′-ACTTCCCTGGCCTATTTGACC-3′
and
(SEQ ID NO: 12)
5′-GGCATGGACGGGTACATCTT-3′;
and
(SEQ ID NO: 13)
HMGCR 5′-CTTGTGGAATGCCTTGTGATTG-3′
and
(SEQ ID NO: 14)
5′-AGCCGAAGCAGCACATGAT-3′.

For miRNA quantification, total RNA was reverse transcribed using the RT2 miRNA First Strand kit (SABiosciences). Primers specific for human miR-33a, miR-611, miR-515-3p (SABiosciences) were used and values normalized to human SNORD44 and SNORD47 as housekeeping genes. For mouse tissues, total RNA from liver, spleen, lung, kidney, brain, heart, aorta and peritoneal macrophages from C57BL6 mice was isolated using the Bullet Blender Homogenizer (Next Advance) in TRIzol. 1 μg of total RNA was reverse transcribed using the QuantiMir kit from System Biosciences Inc (SBI) for miR-33 quantification and normalized to U6 using quantitative PCR as described above.

PCR Array Gene Expression Profiling

Total RNA was extracted from HepG2 or peritoneal macrophages over-expressing either a control mimic or an miR-33 mimic, as described above. Reverse transcription was performed on 1 μg total RNA using the RT2 First Strand kit and quantitative RT-PCR (QRT-PCR) analysis of 84 lipid-metabolism related genes was performed using Lipoprotein Signaling & Cholesterol Metabolism RT2 Profiler PCR Arrays (SABiosciences) as per the manufacturer's protocol. The complete list of the genes analyzed is available online at http://www.sabiosciences.com. Data analysis was performed using the manufacturer's integrated web-based software package for the PCR Array System using ΔΔCₜ based fold-change calculations. Data is the mean of three independent experiments and is represented by fold change compared to control mimic±s.e.m.

Lipid Analysis and Lipoprotein Profile Measurement

Mice were fasted for 12-14 h before blood samples were collected by retro-orbital venous plexus puncture. Plasma was separated by centrifugation and stored at −80° C. Total plasma cholesterol and HDL-cholesterol were enzymatically measured with the Amplex red cholesterol assay kit (Molecular Probes), according to the manufacture's instructions. The lipid distribution in plasma lipoproteins fractions were assessed by fast-performance liquid chromatography (FPLC) gel filtration with 2 Superose 6 HR 10/30 columns (Pharmacia).

Statistics

Data are presented as mean±the standard error of the mean (SEM) (n is noted in the Fig legends), and the statistical significance of differences was evaluated with the Student's t test. Significance was accepted at the level of p<0.05.

Results

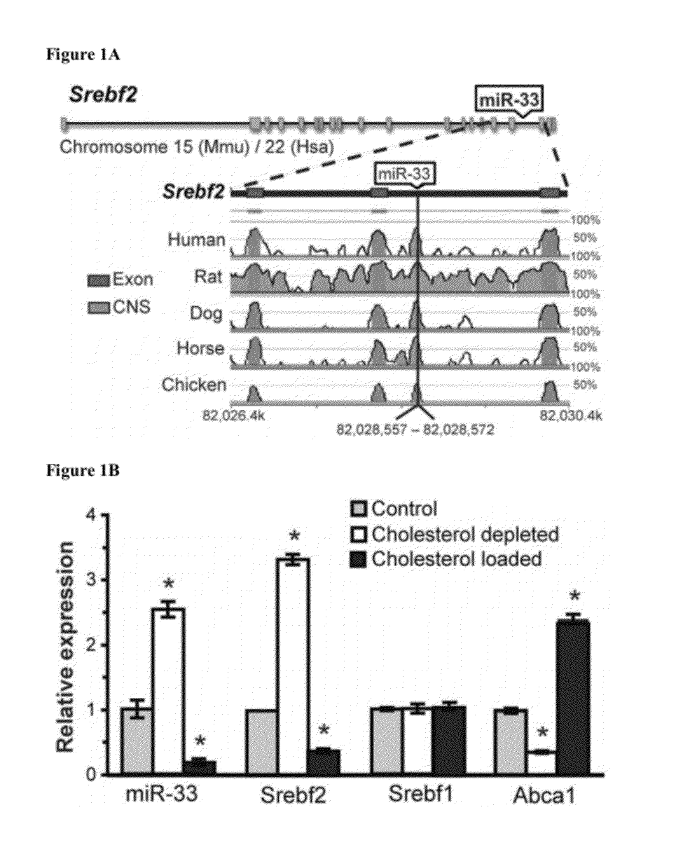

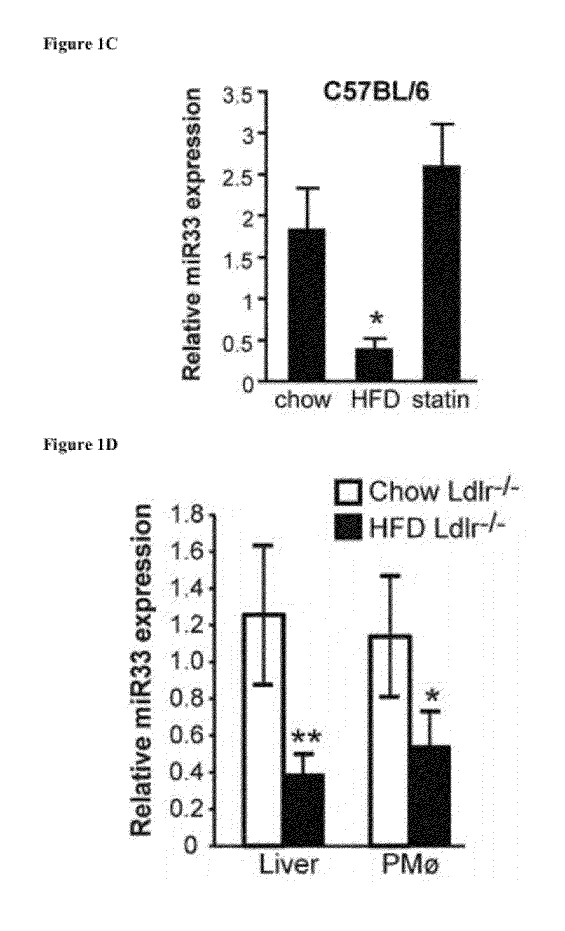

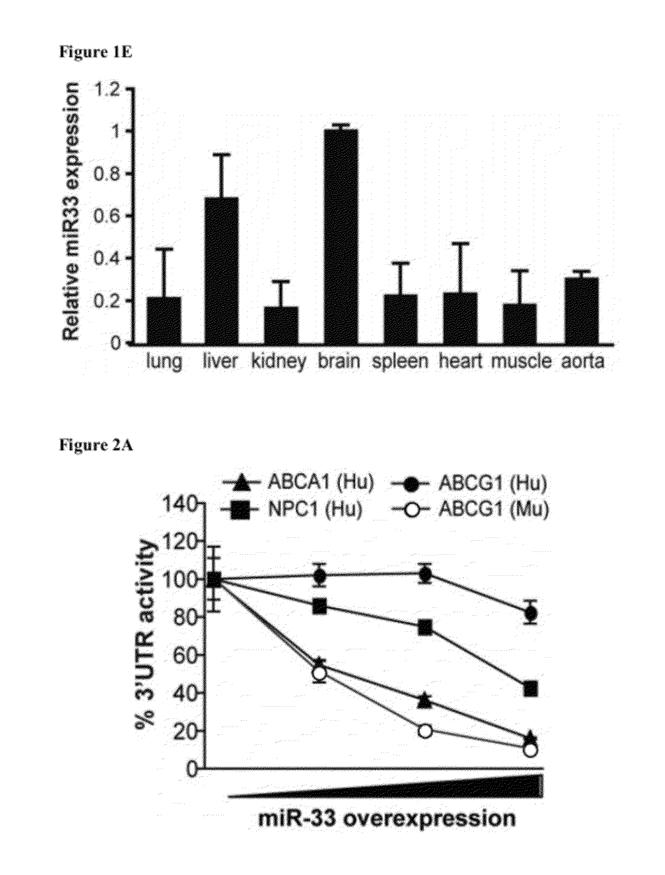

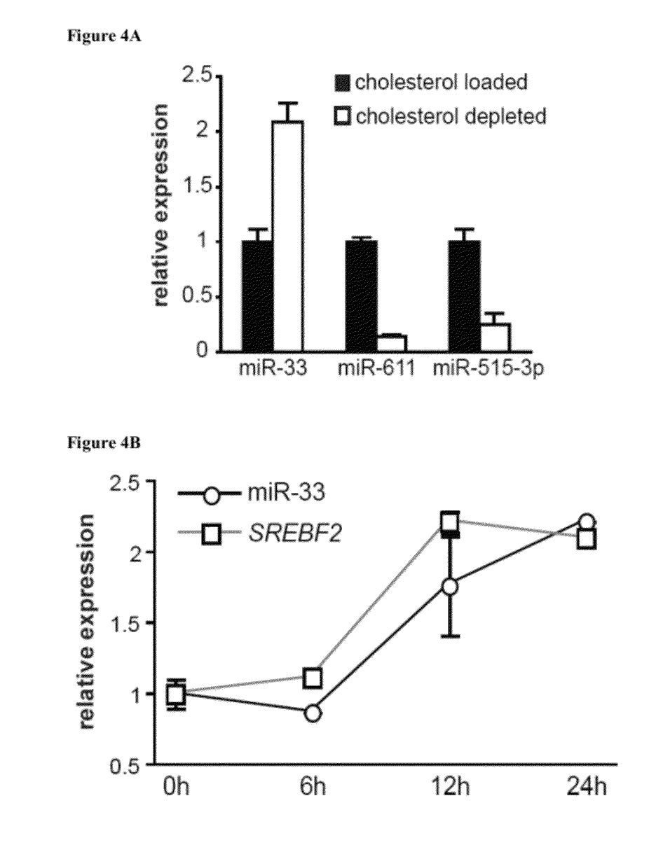

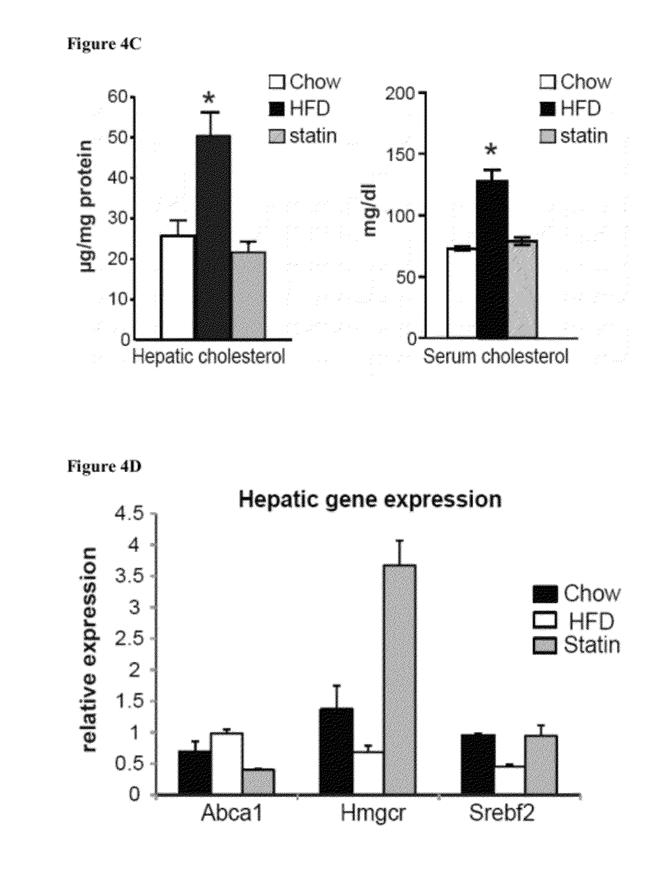

An unbiased genome-wide screen of miRNAs modulated by cellular cholesterol content was performed. The screen revealed a subset of 21 miRNAs that are differentially regulated in human macrophages by cholesterol depletion and cholesterol enrichment, including several whose predicted gene targets are involved in cholesterol uptake, transport and efflux (Table 1). Confirmation of these candidates identified miRNAs that were both up- and down-regulated by cellular cholesterol content (FIG. 4A). Sequence alignment revealed that one of these candidates, hsa-miR-33a and its mouse homologue mmu-miR-33 (herein after referred to as “miR-33”), is encoded within intron 16 of SREBF2, a gene that encodes a key transcriptional regulator of cholesterol uptake and synthesis (FIG. 1A) (Horton J D, et al. J Clin Invest. 2002 109(9):1125-31). The pre-miRNA is highly conserved in mammals (FIG. 1A), prompting the selection of miR-33 for further characterization of its role in cholesterol metabolism.

Expression of miR-33 and SREBF2 was coordinately downregulated in mouse peritoneal macrophages by cholesterol loading, indicating that these gene regulatory elements are co-transcribed (FIG. 1B). Macrophages depleted of cholesterol using the HMG-CoA reductase inhibitor simvastatin showed robust upregulation of both miR-33 and SREBF2, but not SREBF1 (FIG. 1B). Levels of miR-33 inversely correlated with the expression of another cholesterol responsive gene, the cholesterol transporter ABCA1 (FIG. 1B). Analysis of the kinetics of miR-33 induction revealed a concomitant increase in miR-33 and SREBP2 levels with simvastatin treatment, consistent with their co-regulation (FIG. 4B).

TABLE 1
Relative miRNA expression in cholesterol
loaded versus depleted macrophages

| miRNA ID | Fold change | Gene targets |
|---|---|---|
| hsa-let-7c | −3.86 | PPARGC1A |
| hsa-miR-130b | −2.38 | LDLR |
| hsa-miR-29c | −2.27 | NPC1, LRP6, LPL, OSBP |
| hsa-miR-302a | −2.17 | VLDLR, ABCA1 |
| hsa-miR-330-3p | 2.58 | LRP8 |
| hsa-miR-33a | −2.67 | ABCA1, NPC1, ABCG1 |
| hsa-miR-342-5p | −2.43 | |
| hsa-miR-369-5p | −2.14 | ABCG1, LPL |
| hsa-miR-376a | −2.02 | LRP2 |
| hsa-miR-449a | −2.00 | |
| hsa-miR-489 | −2.35 | LIPH, LRP11 |
| hsa-miR-502-5p | −2.58 | LRP6, APOB48R |
| hsa-miR-503 | −4.44 | SRA |
| hsa-miR-515-3p | 2.09 | SCARF1 |
| hsa-miR-518c | −3.21 | LPIN1 |
| hsa-miR-525-3p | −2.58 | FFAR3 |
| hsa-miR-548a-3p | 5.16 | CAV1 |
| hsa-miR-611 | 21.11 | APOE, OSBP2, MARCO |
| hsa-miR-615-3p | −2.09 | APOB |
| hsa-miR-629 | −2.27 | LRP6 |
| hsa-miR-671-5p | −2.09 | LRP6 |

THP-1 cells were treated with AcLDL or Simvastatin for 24 h and relative miRNA expression was analyzed by QRT-PCR miRNA PCR Array. Data are the mean of three independent experiments. Predicted gene targets were identified using Targetscan.

### Example 2

### Demonstration that miR-33 is Regulated by Dietary Cholesterol In Vivo

Materials and Methods

Mice

All animal experiments were approved by the Institutional Animal Care Use Committee of New York University Medical Center. Six-week old C57BL6 and Ldlr^{-/-} mice were obtained from Jackson Laboratory. Ldlr^{-/-} mice were placed on a either a chow diet or a high-fat diet (HFD) containing 0.3% cholesterol and 21% (wt/wt) fat (from Dyets Inc) for 12 weeks. C57BL6 mice were placed on either a chow diet, HFD, or a chow diet containing 0.005% (wt/wt) rosuvastatin (AstraZeneca UK Ltd), equaling 5 mg/kg body weight per day for 3 weeks. At sacrifice, mice were fasted for 12-14 h before blood samples were collected by retro-orbital venous plexus puncture. Liver samples were collected and stored at −80° C. and total RNA was harvested for miRNA and gene expression analysis.

Immunohistochemistry

Snap-frozen fixed liver embedded in optimal cutting temperature (OCT) were sectioned, fixed in 4% PFA, and processed for Dapi staining according to standard protocols.

Results

It was next determined whether miR-33 is regulated under physiologic conditions by measuring its expression in mice fed a chow, rosuvastatin-supplemented or high fat diet. Consistent with in vitro observations, hepatic miR-33 levels correlated inversely with cholesterol levels and Abca1 expression, and positively correlated with Srebf2 mRNA levels, indicating that miR-33 is regulated by dietary cholesterol in vivo (FIG. 1C and FIGS. 4C and 4D).

Levels of miR-33 were also regulated in two mouse models of hypercholesterolemia: Ldlr^{-/-} and Apo^{-/-} mice. Hepatic and peritoneal macrophage miR-33 levels were markedly reduced in Ldlr^{-/-} mice that were fed a high fat diet (FIG. 1D). Similarly, miR-33 levels in peritoneal macrophages from hypercholesterolemic Apoe^{-/-} mice correlated inversely with cellular cholesterol ester content and expression of ABCA1 (FIG. 5B).

Expression of miR-33 was next examined in mouse tissues and various cell lines. In addition to macrophages, miR-33 was highly expressed in mouse and human hepatic cells and to a lesser extent in endothelial cells (FIG. 5C). Furthermore, miR-33 was widely expressed in mouse tissues, and was particularly abundant in the brain and liver (FIG. 1E).

### Example 3

### Identification of MiR-33 Gene Targets

Materials and Methods

miR-33 and Anti-miR-33 Transfection

Mouse peritoneal macrophages, J774, HepG2, Hepa, and EAhy926 were transfected with 40 nM miRIDIAN miRNA mimics (miR-33) or with 60 nM miRIDIAN miRNA inhibitors (anti-miR-33) (Dharmacon) utilizing Oligofectamine (Invitrogen). All experimental control samples were treated with an equal concentration of a non-targeting control mimic sequence (Con miR) or inhibitor negative control sequence (Con Inh), for use as controls for non-sequence-specific effects in miRNA experiments. Verification of miR-33 overexpression and knockdown was determined using qPCR, as described above. Additionally, lentiviral expression clones containing either an miR-33a precursor (miR-33) or an anti-sense to miR-33a (anti-miR-33) and scrambled controls (scr-miR) were obtained from System Biosciences and packaged into lentiviral particles in 293T cells using the pPACKH1 packaging system, with co-expression of copGFP for expression monitoring. Human THP-1 cells were transduced with lentivirus at an MOI of 1:10 and GFP-positive cells were sorted by FACS.

Western blot Analysis

Cell were lysed in ice-cold buffer containing 50 mM Tris-HCl, pH 7.5, 125 mM NaCl, 1% NP-40, 5.3 mM NaF, 1.5 mM NaP and 1 mM orthovanadate, 175 mg/ml octylglucopyranoside and 1 mg/ml of protease inhibitor cocktail (Roche) and 0.25 mg/ml AEBSF (Roche). Cell lysates were rotated at 4° C. for 1 h before the insoluble material were removed by centrifugation at 12000×g for 10 min. After normalizing for equal protein concentration, cell lysates were resuspended in SDS sample buffer before separation by SDS-PAGE. Following overnight transfer of the proteins onto nitrocellulose membranes. The membranes were probed with the indicated antibodies, and protein bands were visualized using the Odyssey Infrared Imaging System (LI-COR Biotechnology). Densitometry analysis of the gels was carried out using ImageJ software from the NIH (http://rsbweb.nih.gov/ij/).

Results

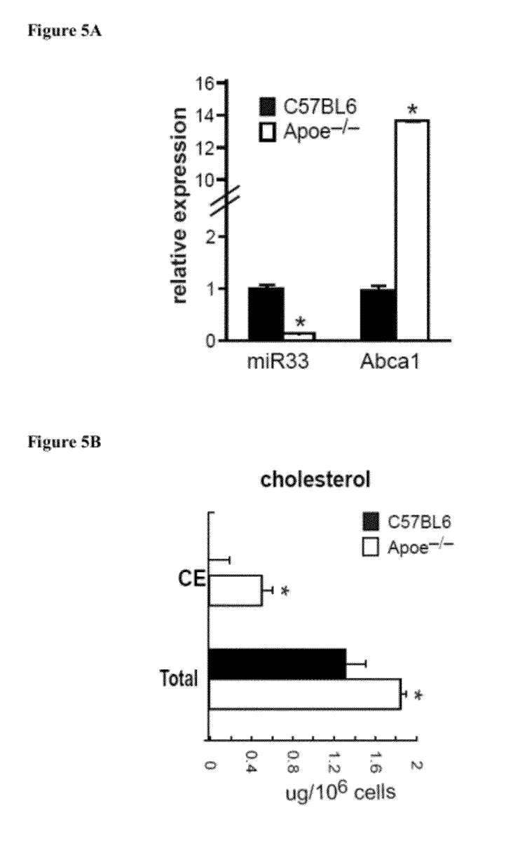

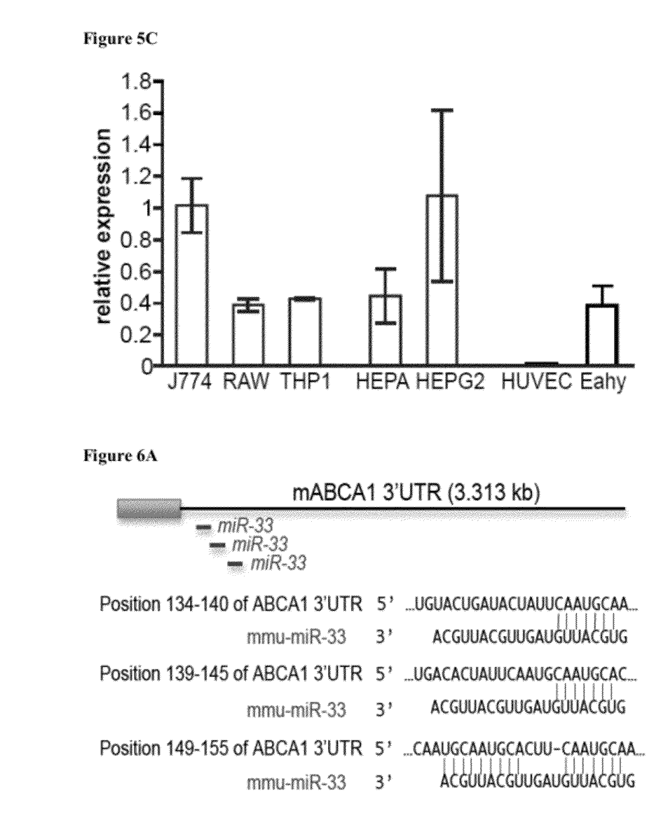

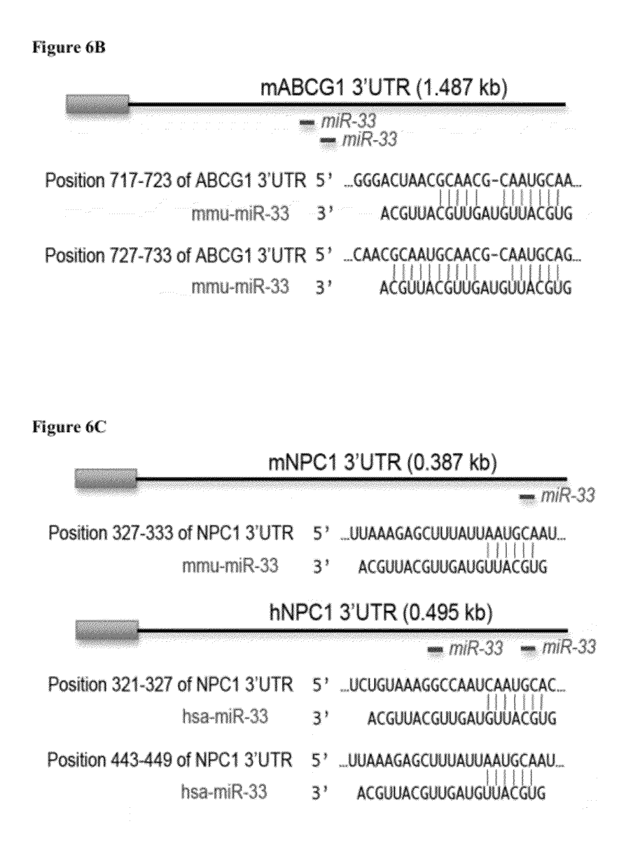

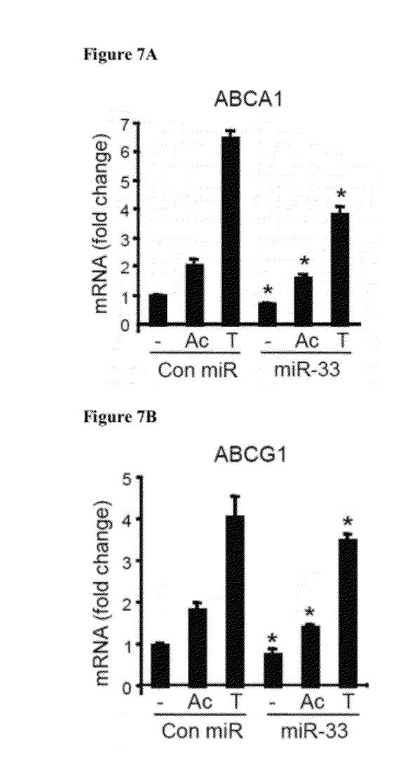

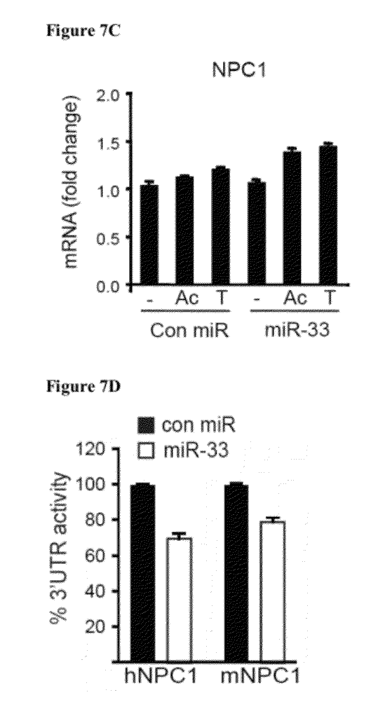

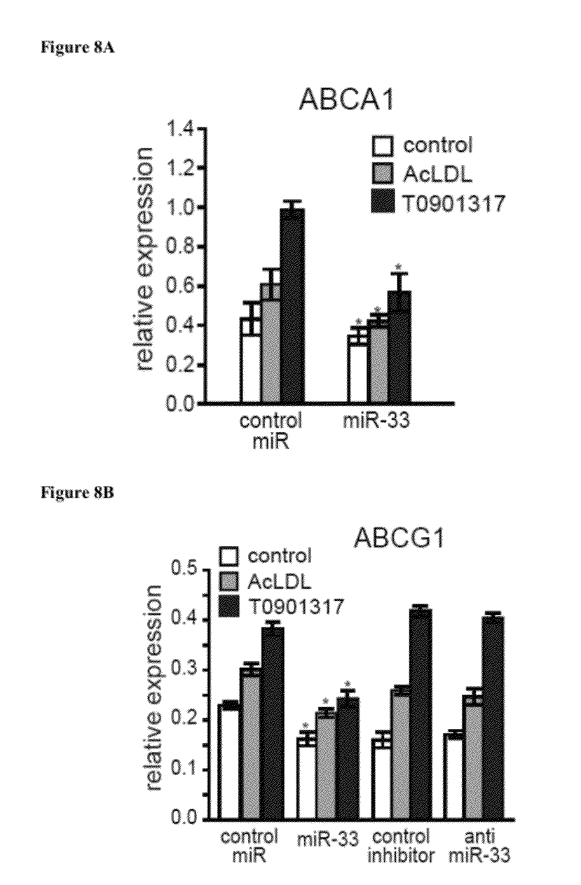

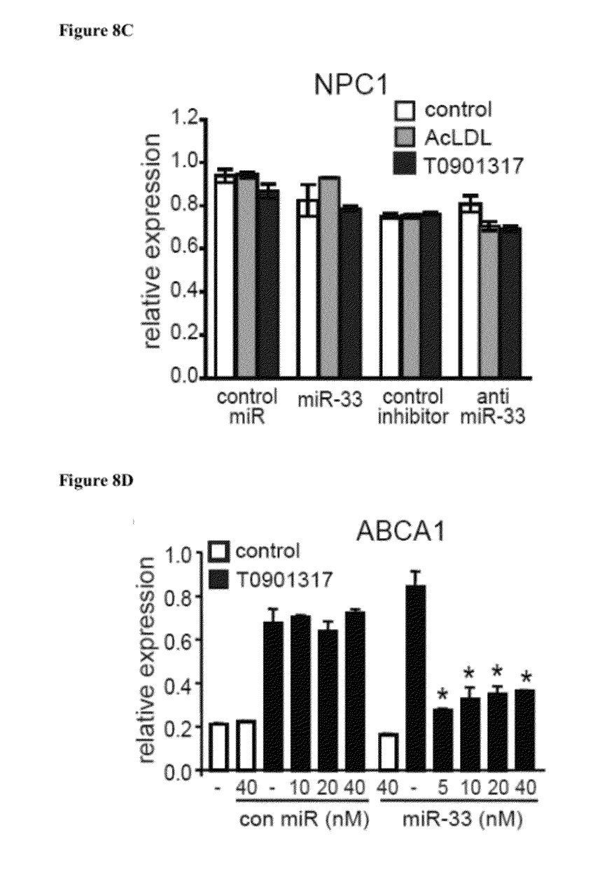

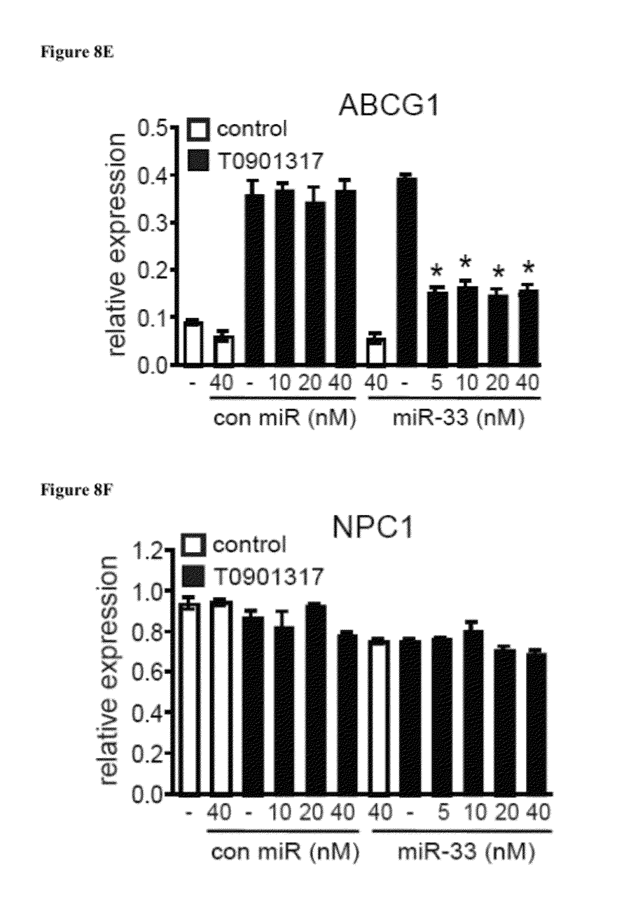

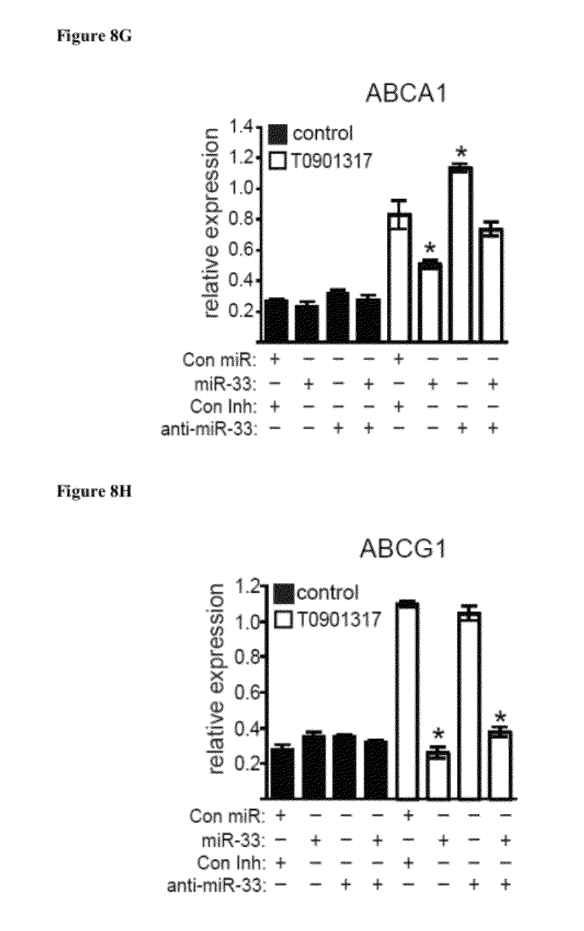

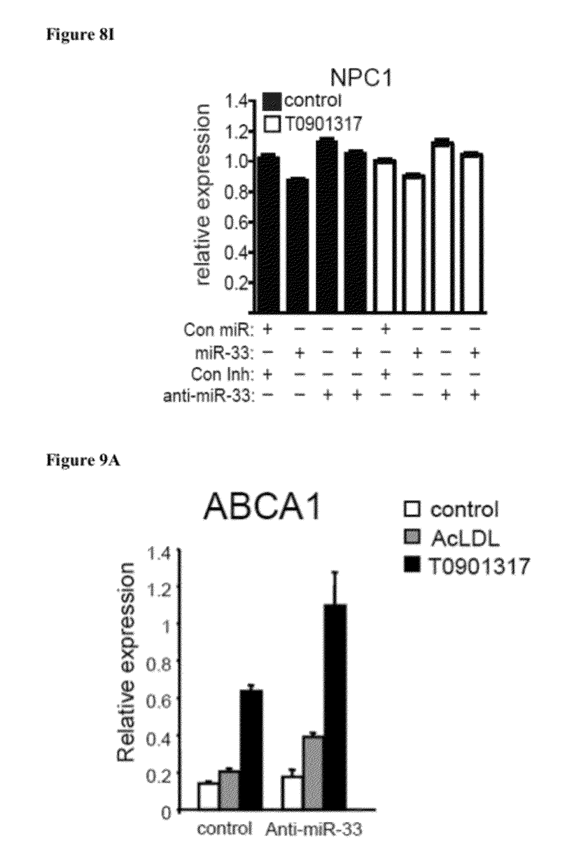

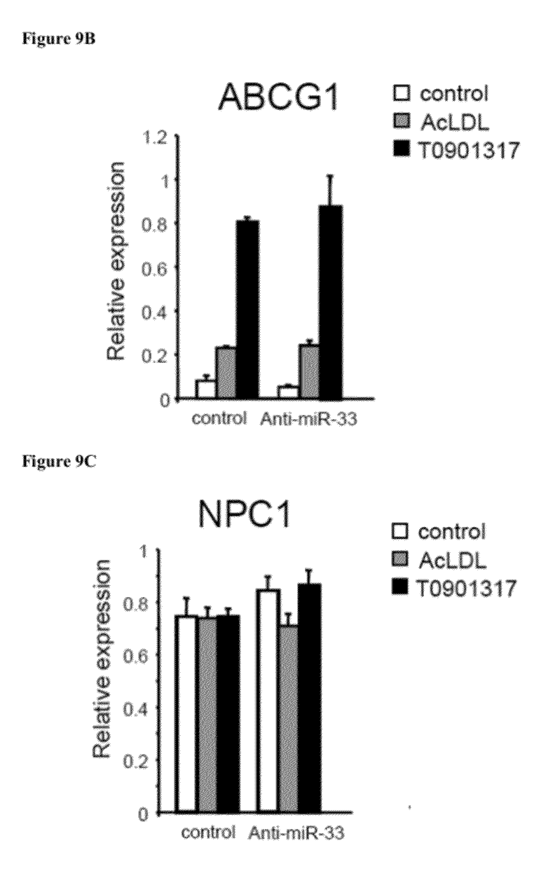

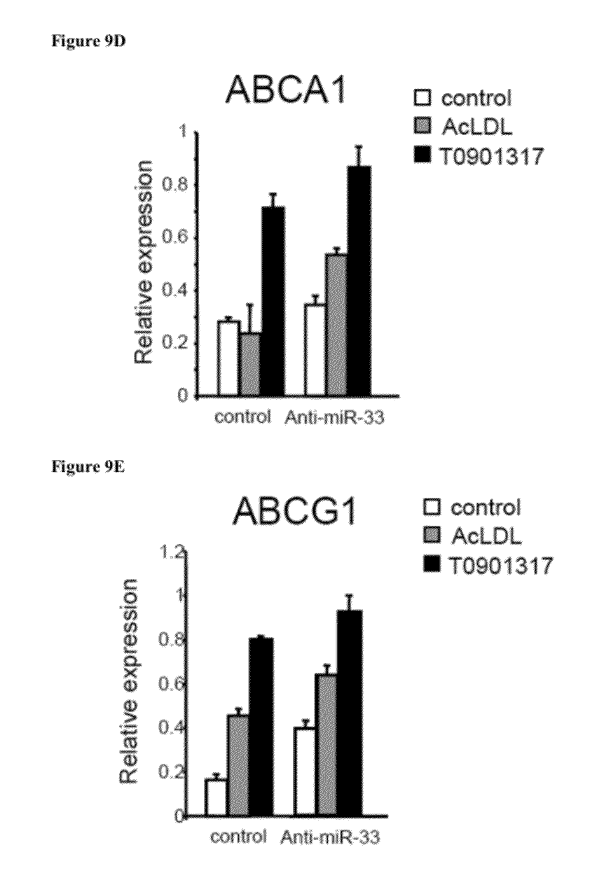

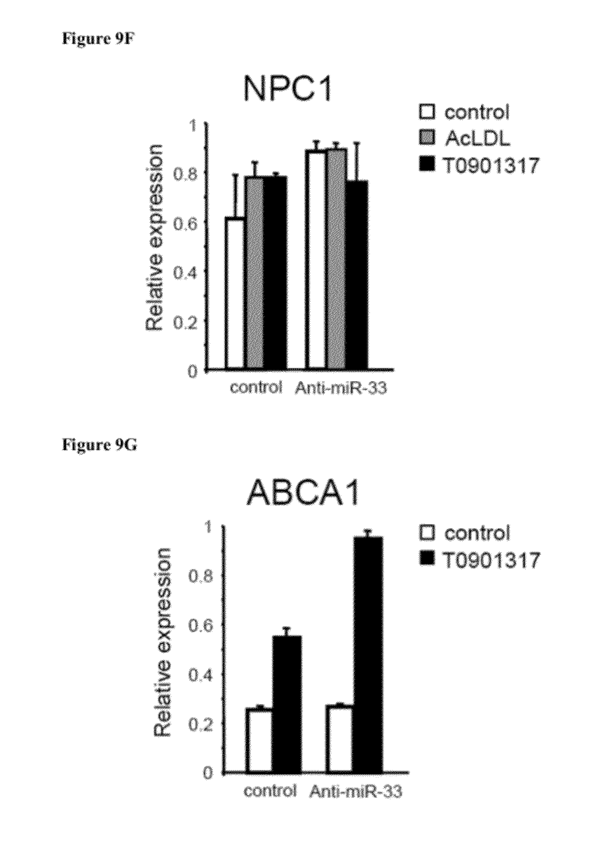

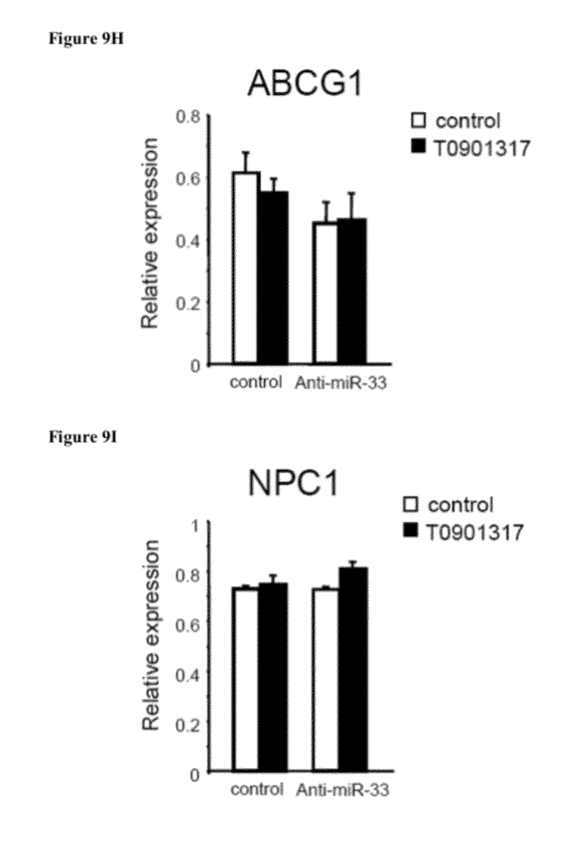

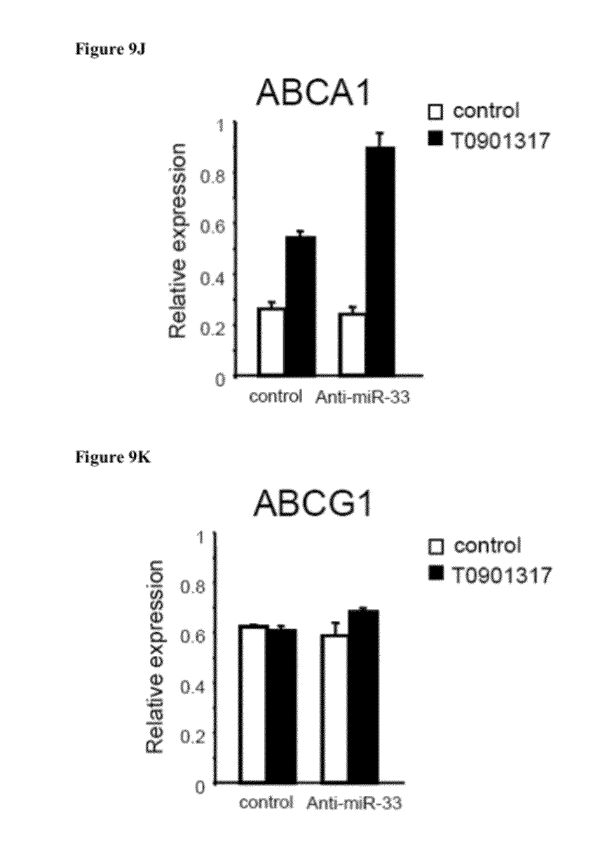

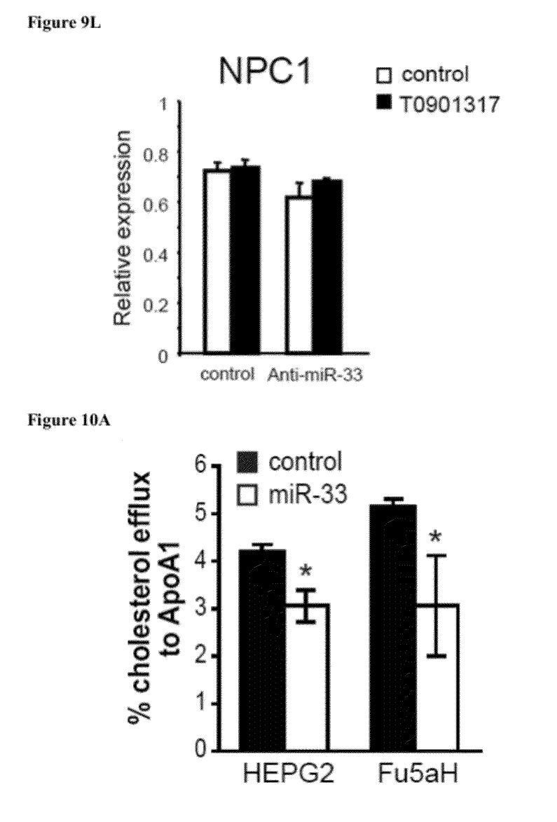

miR-33 and its potential gene targets were analyzed using several miRNA target prediction algorithms (Bartel M, et al. J. Pathol. 2008 215(2):195-203). Putative binding sites were identified for mouse miR-33 in the 3′UTR of several genes involved in cellular cholesterol mobilization including the ABC transporters ABCA1 and ABCG1 and the endolysosomal transport protein NPC1 (FIG. 6), indicating that miR-33 coordinates cholesterol homeostasis through these pathways (Ory DS. Trends Cardiovasc Med. 2004 14(2):66-72; Tall A R, et al. Cell Metab. 2008 7(5):365-75). The effect of miR-33 on expression of ABCA1, ABCG1 and NPC1 in macrophages treated with AcLDL (to cholesterol enrich) or the LXR ligand T0901317 (to directly stimulate expression of the three genes) was determined (Beaven S W, et al. Annu Rev Med. 2006 57:313-29). Transfection of mouse peritoneal macrophages with miR-33, (but not a control miRNA) strongly decreased the stimulation of both ABCA1 and ABCG1 protein and mRNA (FIGS. 7A, 7B, 8A, 8B). These effects of miR-33 were seen with as little as 5 nM (FIGS. 8D, 8E) and were reversible by co-incubation with an anti-sense inhibitor of miR-33 (antimiR-33), (FIGS. 8G, 8H). miR-33 also repressed ABCA1 and ABCG1 protein in mouse hepatic cells indicating that its effects are not cell type specific. Notably, inhibition of endogenous miR-33 by antimiR33 increased the expression of ABCA1 and ABCG1 in macrophages, and ABCA1 in hepatocytes, consistent with the hypothesis that miR-33 has a physiological role in regulating the expression of these transporters (FIG. 9).

While miR-33 comparably repressed ABCA1 in cells of mouse and human origin, this was not the case for NPC1 and ABCG1. miR-33 strongly suppressed NPC1 protein in cells of human origin, whereas in mouse cells, miR-33 suppressed NPC1 protein levels only modestly and had no effect on Npc1 mRNA levels (FIG. 7C). Furthermore, transfection of human macrophage, hepatic and endothelial cells with miR-33 had no detectable effect on ABCG1 protein. Conservation map analysis revealed that while the predicted miR-33 target sites in the 3′UTR of mouse ABCA1 and NPC1 are highly conserved (FIG. 6), the putative sites for miR-33 in the 3′UTR of ABCG1 are present only in mouse and rat. Moreover, a second miR-33 binding site was identified in human NPC1 (FIG. 6C) highlighting the species-specific regulation of cholesterol metabolism genes by miR-33.

### Example 4

### Assessment of the Effects of miR-33 on the 3′UTR of Human and Mouse ABCA1, ABCG1, and NPC1

Materials and Methods

3′UTR Luciferase Reporter Assays

cDNA fragments corresponding to the entire 3′UTR of hABCA1, hNPC1, mNPC1 and hABCG1 were amplified by RT-PCR from total RNA extracted from HepG2 cells with XhoI and NotI linkers. Additionally, 3′UTR of mABCG1 was cloned using cDNA from mouse liver with the same strategy. The PCR products were directionally cloned downstream Renilla luciferase open reading frame of the psiCHECK2™ vector (Promega) that also contains a constitutively expressed firefly luciferase gene, which is used to normalize transfections. Point mutations in the seed region of predicted miR-33 sites within the 3′UTR of hABCA1, hNPC1 and mABCG1 were generated using Multisite-Quickchange (Stratagene) according to the manufacturer's protocol. All constructs were confirmed by sequencing. COS-7 cells were plated into 12-well plates (Costar) and co-transfected with 1 μg of the indicated 3′UTR luciferase reporter vectors and the miR-33 mimic or negative control mimic (Dharmacon) utilizing Lipofectamine 2000 (Invitrogen). Luciferase activity was measured using the Dual-Glo Luciferase Assay System (Promega). Renilla luciferase activity was normalized to the corresponding firefly luciferase activity and plotted as a percentage of the control (cells co-transfected with the corresponding concentration of control mimic). Experiments were performed in triplicate wells of a 12-well plate and repeated at least three times.

The following are the point mutations in the miR-33 sites within the

3′UTR of hABCA1, hNPC1 and mABCG1:
(SEQ ID NO: 15)
hABCA1-3′UTR, 5′-TCAATGCAATGCAATTCAATGC-3′,
(SEQ ID NO: 16)
hABCA1-3′UTR-PM1, 5′-TCTTTGTAATGCAATTCAATGC-3′,
(SEQ ID NO: 17)
hABCA1-3′UTR-PM2, 5′-TCTTTGTAATGCAATTCTATGT-3′,
(SEQ ID NO: 18)
hNPCA1-3′UTR, 5′-AATCAATGCACTGTCTGT-3′,
(SEQ ID NO: 19)
hNPCA1-3′UTR-PM, 5′-AATCTTTGCACTGTCTGT-3′,
(SEQ ID NO: 20)
MABCG1-3′UTR, 5′-CGCAATGCAACGCAATGC-3′,
(SEQ ID NO: 21)
MABCG1-3′UTR-PM1, 5′-CGCTTTGCAACGCAATGC-3′,
and
(SEQ ID NO: 22)
MABCG1-3′UTR-PM2, 5′-CGCTTTGCAACGCTTTGC-3′.

### Results

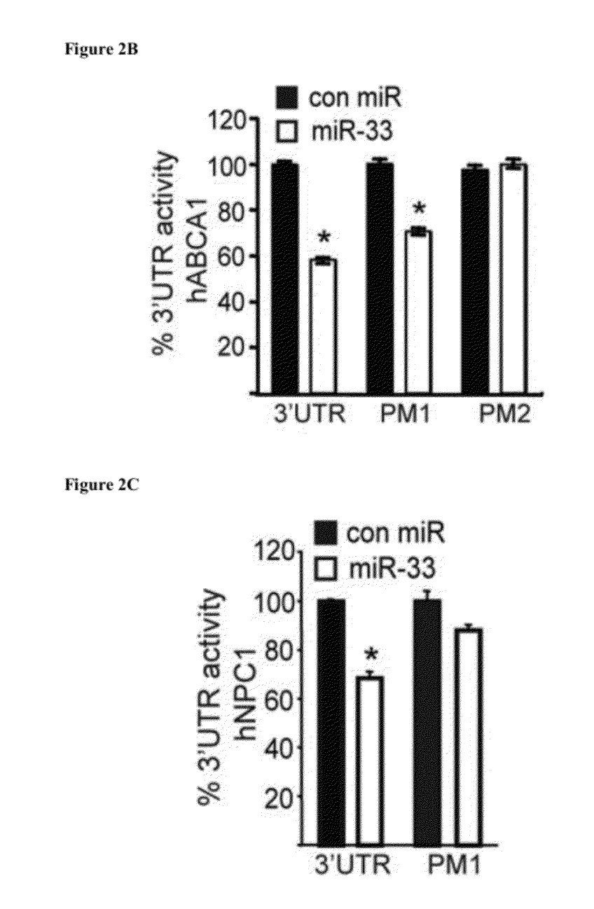

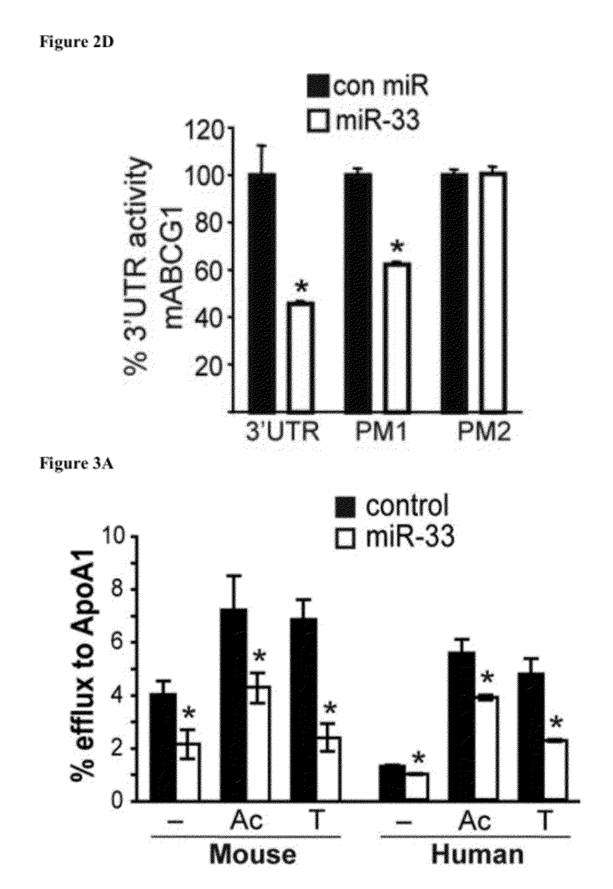

To assess the effects of miR-33 on the 3′UTR of human and mouse ABCA1, ABCG1, and NPC1, luciferase reporter constructs were used. miR-33 markedly repressed mouse, but not human, ABCG1 3′UTR activity (FIG. 2A). Furthermore, consistent with species-conserved miR-33 target sites, miR-33 significantly inhibited hABCA1 and hNPC1 3′UTR activity (FIG. 2A). Mutation of the miR-33 target sites relieved miR-33 repression of hABCA1-, mABCG1- and hNPC1-3′UTR activity, consistent with a direct interaction of miR-33 with these sites (FIG. 2B to 2D). Of note, mutation of both miR-33 sites in the 3′UTR of hABCA1 and mABCG1 was necessary to completely reverse the inhibitory effects of miR-33 (FIGS. 2B and 2D). miR-33 more strongly repressed hNPC1 compared to mNPC1-3′UTR activity, consistent with the presence of an additional miR-33 binding site (FIG. 7D). Together, these experiments identify ABCA1 and NPC1 as conserved targets of miR-33, whereas ABCG1 is a target only in mouse.

### Example 5

### Increased Cholesterol Efflux in Both Macrophages and Hepatocytes by MiR-33 Inhibitor

Materials and Methods

Cholesterol Efflux Assays

THP-1 cells expressing either a control lentiviral construct, a pre-miR33a lentiviral construct or an anti-miR-33 lentiviral construct (System Biosciences Inc.) were seeded with PMA in 24-well plates at a density of 1×10⁶ cells per well three days prior to stimulation. On day 3, cells were labeled with 0.5 μCi/ml of 3H-cholesterol (PerkinElmer, Waltham, Mass.) for 24 hours. J774 macrophages, HepG2 or Fu5aH hepatocytes were transfected with either a control mimic, an miR-33 mimic, a control inhibitor or a anti-miR-33 inhibitor (Dharmacon) and seeded at a density of 1×10⁶ cells per well one day prior to loading with 0.5 μCi/ml ³H-cholesterol for 24 hours. Then, cells were washed twice with PBS and incubated with 2 mg/ml fatty-acid free BSA (FAFA, Sigma) in media for 1 hour prior to addition of 50 μg/ml human apoA1 or 50 μg/ml HDL in FAFA-media with or without the indicated treatments. For cholesterol depleted experiments, cells were treated with 5 μM simvistatin overnight prior to addition of apoA1. Supernatants were collected after 6 hours and expressed as a percentage of total cell ³H-cholesterol content (total effluxed ³H-cholesterol+cell-associated ³H-cholesterol).

Results

To confirm the specificity of miR-33 targeting of ABCA1, ABCG1 and NPC1, the effect of miR-33 overexpression on other lipid metabolism-related genes was assessed in macrophages and hepatocytes. Whereas ABCA1 was predictably downregulated in these cells, few changes were observed in the expression of non-miR-33 targets. Other genes containing putative miR-33 binding sites such as HMGCR and SCAP were modestly downregulated at the mRNA level, but there was no detectable change in protein expression.

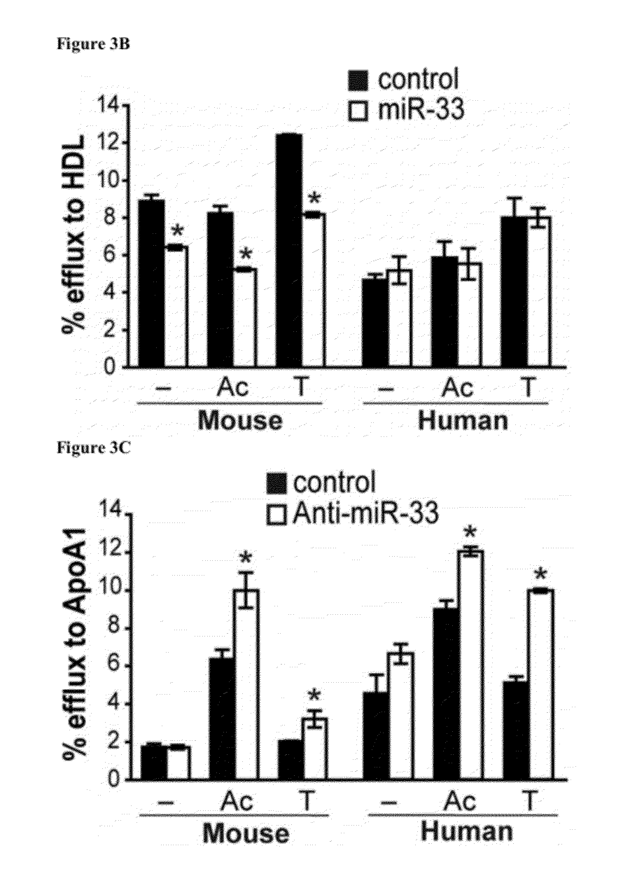

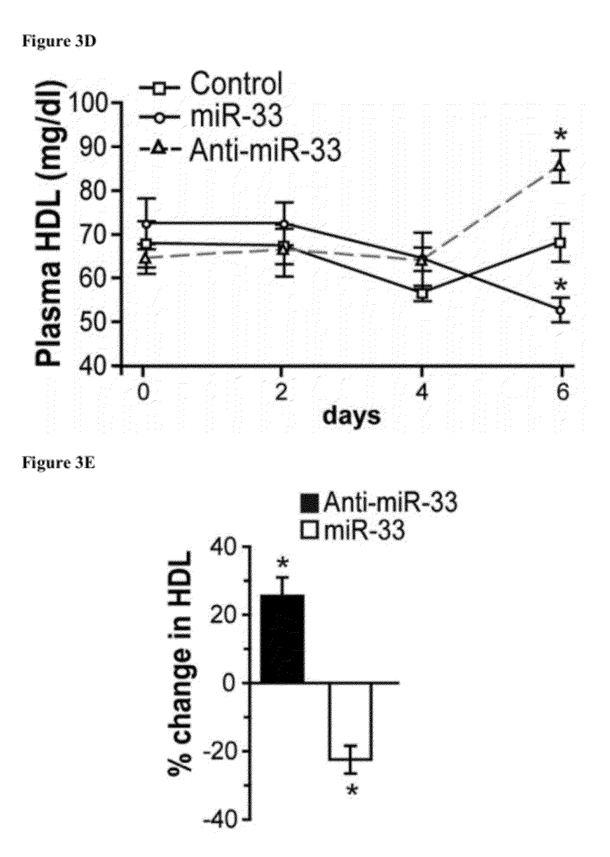

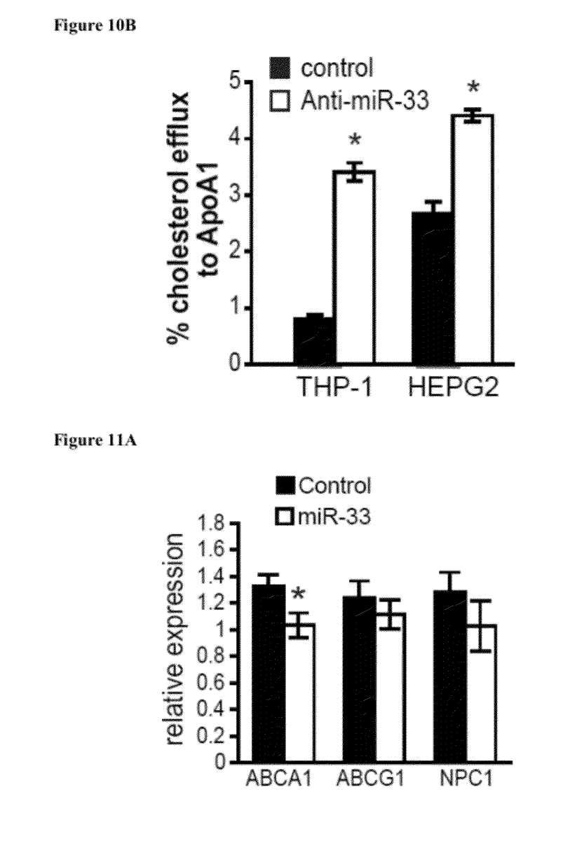

The ability of ABCA1 and ABCG1 to stimulate the efflux of cholesterol from cells in the periphery, particularly cholesterol-laden macrophages in atherosclerotic plaques, is an important anti-atherosclerotic mechanism (Tall A R, et al. Cell Metab. 2008 7(5):365-75). Transfection of J774 murine macrophages with miR-33 attenuated cholesterol efflux to apolipoprotein A1 (apoA1) and HDL, in agreement with the known functions of ABCA1 and ABCG1, respectively (FIGS. 3A and 3B). miR-33 did not impair cholesterol efflux to HDL in human THP-1 macrophages, consistent with the lack of miR-33 binding sites in the human ABCG1 3′UTR (FIG. 3B). Similar results were seen in human and rat hepatocytes where transfection of miR-33 reduced cholesterol efflux to apoA1 (FIG. 10A). Notably, antagonism of endogenous miR-33 increased ABCA1 protein and cholesterol efflux to apoA1 in both murine and human macrophages (FIG. 3C). Importantly, under conditions in which miR-33 is increased (i.e. cholesterol depletion), anti-miR-33 significantly increased cholesterol efflux in both macrophages and hepatocytes (FIG. 10B). Thus, manipulation of cellular miR-33 levels alters macrophage cholesterol efflux, a critical first step in the reverse cholesterol transport pathway for the delivery of excess cholesterol to the liver (Tall A R, et al. Cell Metab. 2008 7(5):365-75).

### Example 6

### In Vivo Overexpression of miR-33 Results in a Progressive Decline of Plasma HDL

Materials and Methods

Lentivirus and Gene Transfer

A lentivirus encoding the miR-33 precursor (miR-33) or an anti-sense to miR-33 (anti-miR-33) and scrambled controls (scr-miR) were obtained from System Biosciences Inc (SBI). For in vivo gene delivery, mice were injected with 2×10⁹ pfu/mouse of each lentiviral construct in 100 μl PBS via retroorbital injection. At sacrifice, liver protein and RNA was harvested and stored at −80° C. A total of 1 ug of total RNA was reverse transcribed and used for PCR Array gene expression profiling as described above from three mice per group (control-miR, miR-33 and anti-miR33). Protein was analyzed by Western blots detecting ABCA1, ABCG1, NPC1, HMGCR, SR-B1, CD36, and HSP90.

Results

In addition to cellular cholesterol efflux, ABCA1 is responsible for initiating HDL formation in the liver (Oram J F, et al. Curr Opin Lipidol. 2000 11(3):253-60). Thus, the effect of manipulating miR-33 levels was investigated in vivo in mice using lentiviruses encoding pre-miR-33, anti-miR33, or control. Efficient lentiviral delivery was confirmed by measuring GFP in the liver.

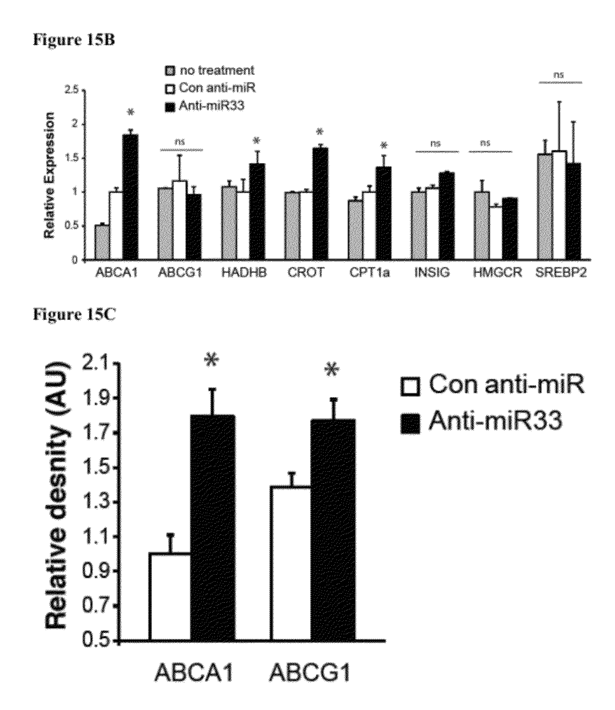

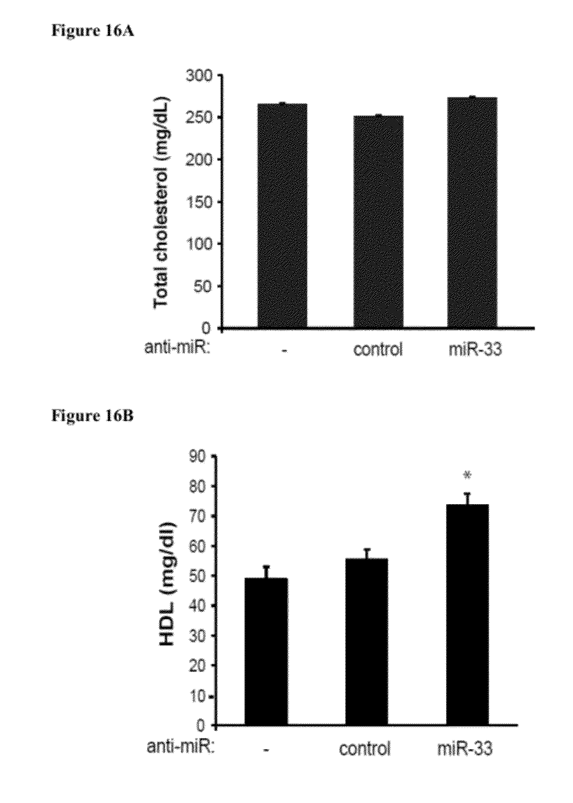

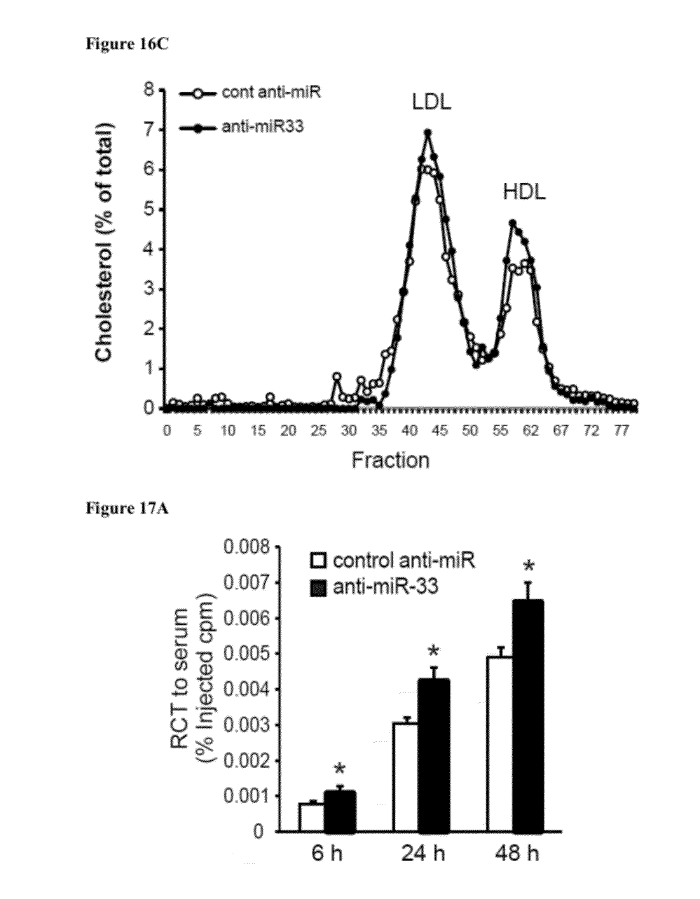

To determine the efficacy of anti-miR33 treatment, the expression of miR-33 and its target genes were measured in the livers of mice after 4 weeks of treatment. Levels of miR-33 detected by quantitative RT-PCR were decreased by more than 60% in anti-miR33 treated mice compared to mice receiving control anti-miR (FIG. 15A).

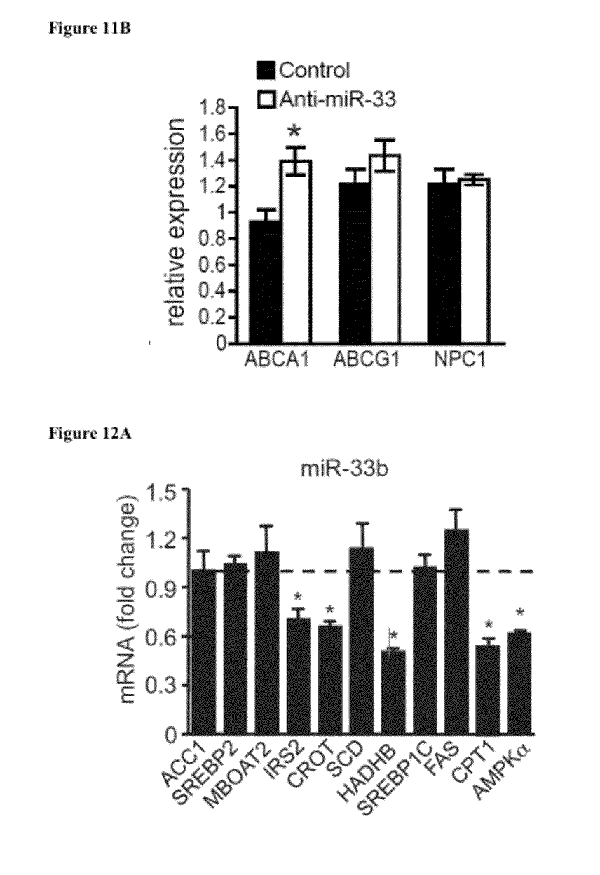

Consistent with this, the expression of ABCA1 in the liver was increased or “de-repressed” in the anti-miR33 treated group compared to untreated or control anti-miR treated mice, however no change in ABCG1 mRNA was observed (FIGS. 11A, 15B). Furthermore, both ABCA1 and ABCG1 protein was increased in the livers of anti-miR33 treated mice, compared to control groups (FIG. 15C). It modestly decreased ABCG1 and NPC1 protein levels. No changes in SR-B1, a cognate receptor for HDL in the liver (Krieger M. J Clin Invest. 2001 September; 108(6):793-7), or other cholesterol-related genes were observed. Moreover, an unbiased assessment of hepatic gene expression revealed few significant differences in the expression of other cholesterol metabolism-related genes in mice treated with miR-33 or anti-miR-33 lentiviruses. Importantly, in vivo overexpression of miR-33 resulted in a progressive decline of plasma HDL as expected by the requirement of ABCA1 for HDL formation (FIG. 3D), with a 22% decrease achieved after 6 days (FIG. 3E). Conversely, mice expressing anti-miR-33 showed a 50% increase in hepatic ABCA1 protein and a concomitant 25% increase in plasma HDL levels after 6 days (FIGS. 3D and 3E). Thus, manipulation of miR-33 levels in vivo alters ABCA1 expression and mobilization of cholesterol to HDL.

Putative binding sites for miR-33 were identified in the 3′UTR of several genes involved in fatty acid metabolism, including carnitine O-octaniltransferase (CROT), Carnitine palmitoyltransferase 1A (CPT1a) and hydroxyacyl-CoA-dehydrogenase (HADHB).

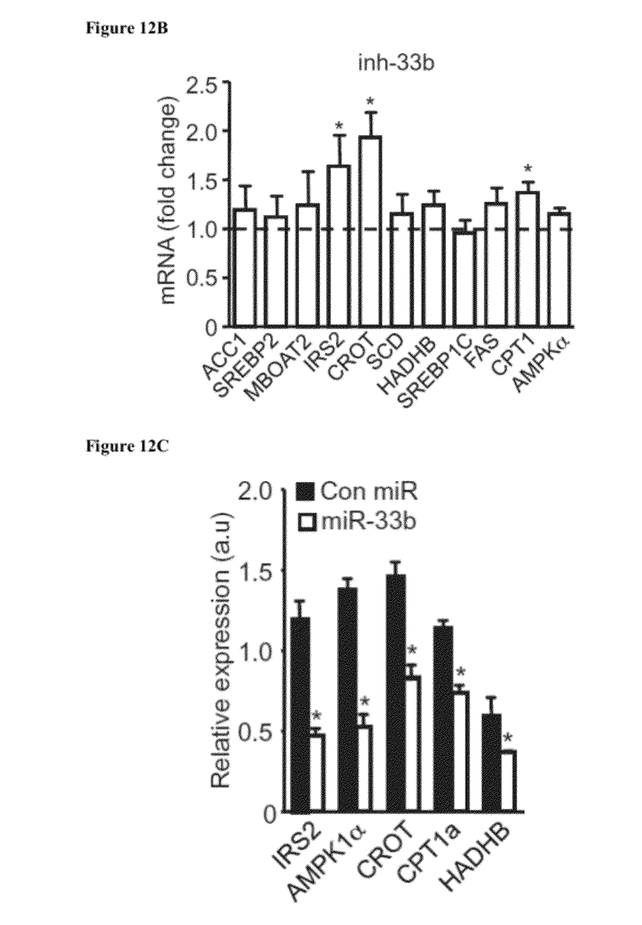

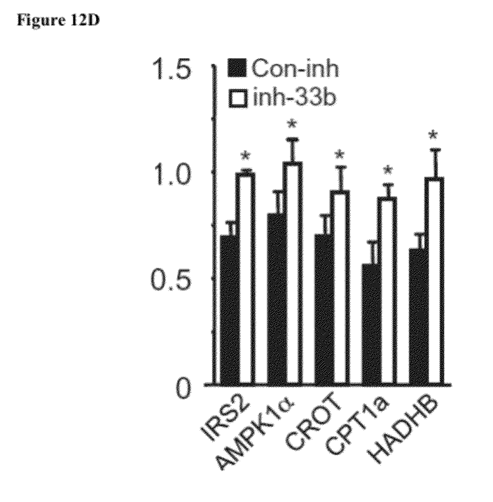

Transfection of Huh7 cells with miR-33b (32-fold increase expression) significantly inhibited the mRNA levels of CROT, CPT1a, HADHB, AMPKα, and IRS2 (FIG. 12A). Notably, inhibition of endogenous miR-33b using anti-miR-33b oligonucleotides (threefold decrease expression) increased the mRNA expression of CROT, CPT1a, AMPKα, and IRS2 in Huh7 cells (FIG. 12B), consistent with a physiological role for miR-33b in regulating the expression of these genes. Similar regulation of these genes by miR-33b was also seen at the protein level (FIGS. 12C and 12D).

The effect of manipulating miR-33 levels in vivo was investigated in mice using lentiviruses encoding premiR-33, anti-miR-33, or a control. Consistently, miR-33 reduced hepatic CROT, HADHB, CPT1a, IRS2, and ABCA1 mRNA expression. Conversely, mice expressing anti-miR-33 showed a modest increase of CROT, CPT1a, IRS2, and ABCA1 mRNA expression, although the effect was not statistically significant.

To show the specificity of miR-33b targeting of CROT, CPT1a, HADHB, AMPKα, and IRS2, the effect of miR-33b overexpression was assessed in HepG2 cells on other fatty acid metabolism related genes using an array that included 84 genes involved in fatty acid transport and biosynthesis, ketogenesis, and ketone body metabolism. Whereas CROT, CPT1a, and AMPKα were predictably down-regulated by miR-33, no changes were observed in the expression of non-miR-33 targets. Furthermore, other genes containing putative miR-33 binding sites such as citrate synthase (CS) and HMGCR were not affected either at the mRNA level or by 3′ UTR activity.

miR-33a and miR-33b have identical seed sequences but differ in 2 of 19 nt of the mature RNA. To determine whether miR-33a and miR-33b have similar effects on CROT, CPT1a, HADHB, AMPKα, Sirtuin 6 (SIRT6), and IRS2 protein expression, Huh7 cells were transfected with a control miR, miR-33a, or miR-33b. miR-33a and miR-33b inhibited CROT, CPT1a, HADHB, AMPKα, S1RT6, and IRS2 protein expression to a similar extent. In addition, both miR-33a and miR-33b significantly inhibited the 3′ UTR activity of Crot, Cpt1a, Hadhb, Ampkα, and Irs2 with only modest differences, indicating that the 2-nt variation in the mature forms of miR-33a and miR-33b does not appreciably affect the gene targeting by these miRNAs.

Altogether, these data indicate that miR-33 is an important regulator of cholesterol metabolism, fatty acid oxidation and insulin signaling. High levels of miR-33 reduce HDL biogenesis and β-oxidation of fatty acid, and impair insulin signaling, three hallmarks of the metabolic syndrome. Thus, strategies that inhibit miR-33 in vivo, such as the use of anti-sense oligonucleotides, can improve these three components of metabolic syndrome (i.e., increase HDL and insulin signaling, and reduce VLDL and triglyceride levels). Anti-miR-33 can also be useful for treating insulin resistance and non-alcoholic hepatic steatosis (fatty liver).

### Example 7

### miR-33 Inhibits Cellular Fatty Acid Oxidation

Materials and Methods

Fatty Acid β-Oxidation Assays

HepG2 and Huh7 cells were transfected with miR-33 (20 nM), anti-miR-33 (40 nM), or negative control (con-miR and con-inh, respectively), and the rate of fatty acid oxidation was determined. Cells were cultured in the presence of [¹⁴C]-oleate (Perkin-Elmer)-BSA complex, and the released [¹⁴C]-carbon dioxide was trapped for 1 h at 37° C. onto filter paper soaked in 100 mM sodium hydroxide. The rate of β-oxidation was calculated as the amount of trapped [¹⁴C]-carbon dioxide in relative units produced per milligram of protein per hour. Experiments were performed in triplicate in p60 plates and were repeated at least three times.

Fly Stocks

Upstream activating sequence (UAS)-DsRed transgenic flies were obtained from the Bloomington Drosophila Stock Center (stock number 6280). UAS-DsRed-miR-33 was generated by cloning the miR-33 hairpin and ˜150-bp flanking sequence downstream of DsRed in the UAST-DsRed plasmid, and transgenic animals were generated by P-element-mediated transgenesis (Bestgene).

Results

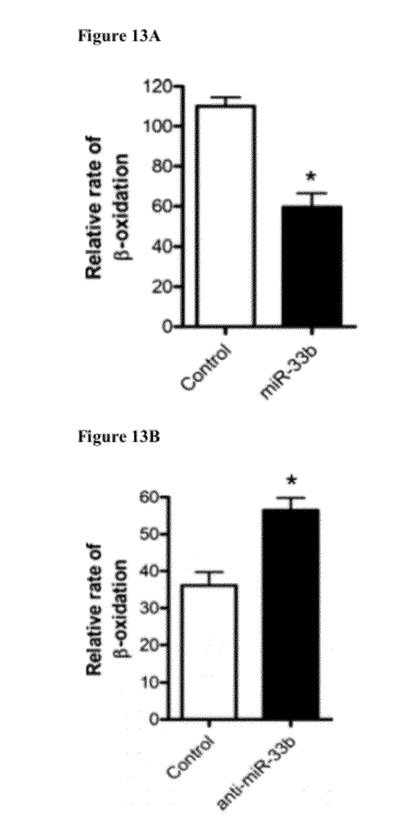

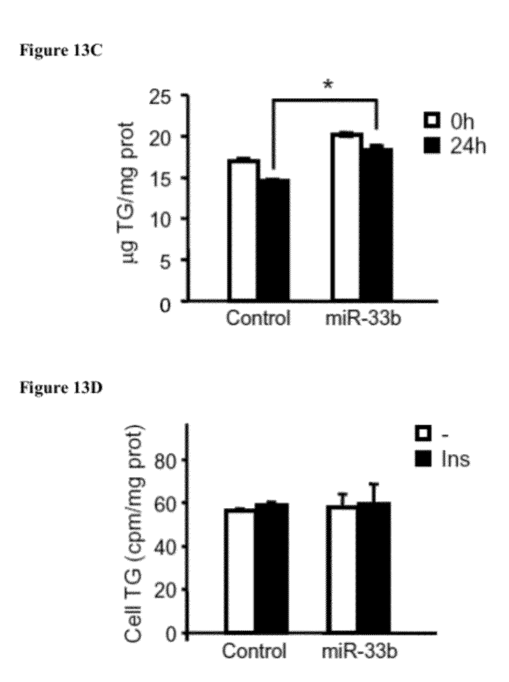

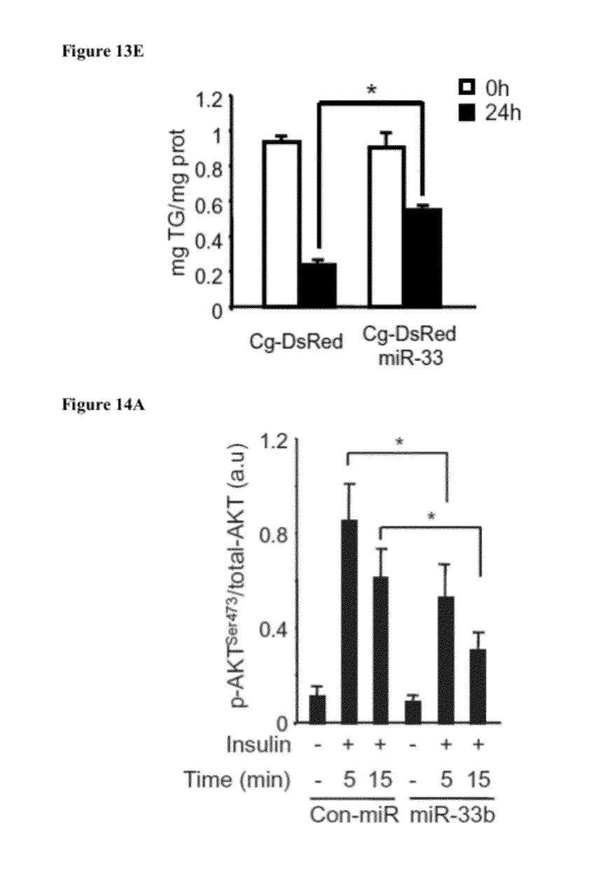

To evaluate the effects of miR-33a and miR-33-b on fatty acid β-oxidation, the release of [¹⁴C]-carbon dioxide from the oxidation of [¹⁴C]-oleate was measured. miR-33b overexpression (27-fold increase) markedly reduced the fatty acid β-oxidation in Huh7 (FIG. 13A) cells. Conversely, inhibition of endogenous miR-33b expression using anti-miR-33 (2.6-fold increase) increased the rate of fatty acid β-oxidation (FIG. 13B). The accumulation of neutral lipids and lipid droplet formation was next evaluated tin Huh7 cells incubated with oleate for 24 h and then starved for the next 24 h. In agreement with the reduced fatty acid β-oxidation rates observed in hepatic cells overexpressing miR-33b, Huh7 cells transfected with miR-33b accumulated more triglycerides (TAG) in larger lipid droplets (FIG. 13C). The increase in triglyceride content was independent of changes in triglyceride synthesis rates. As seen in FIG. 13D, miR-33 expression did not alter basal and insulin-induced triglyceride synthesis.

In order to determine if miR-33 plays a role in maintaining lipid homeostasis in Drosophila, a transgenic fly was generated that overexpressed miR-33 in the fat body. Fat bodies from starved miR-33 transgenic flies, dissected and stained with Bodipy, showed large lipid droplets in many cells compared with control flies. Similarly, flies overexpressing miR-33 accumulated more TAG upon starvation (FIG. 13E), indicating that the function of miR-33 in regulating fatty acid oxidation is conserved from Drosophila to humans.

### Example 8

### miR-33 Regulates Insulin Signaling

Materials and Methods

miR-33 and Anti-miR-33 Transfection.

HepG2 and Huh7 cells were transfected with 40 nM miRIDIAN miRNA mimics (miR-33) or 60 nM miRIDIAN miRNA inhibitors (anti-miR-33) (Dharmacon) using Oligofectamine (Invitrogen). All experimental control samples were treated with an equal concentration of a nontargeting control mimic sequence (CM) or an inhibitor negative control sequence (CI) to control for nonsequence-specific effects in miRNA experiments

AKT Kinase Assay

The in vitro nonradioactive AKT kinase assay was performed using immobilized AKT (1G1) mAb to immunoprecipitate AKT from cell extracts and the GSK-3 fusion protein as a substrate. Phosphorylation of GSK-3 was measured by Western blotting using Phospho-GSK-3 (Ser219) antibody (Cell Signaling).

2-Deoxyglucose Uptake

Huh7 cells were transfected with miR-33b (20 nM) or negative control (Con-miR) for 48 h. Then, cells were cultured in serum-free media for 24 h, washed two times, and placed into warmed buffer for 2 h before glucose uptake studies. Insulin (100 nM) was added for 10 min. After this, 0.5 mCi/mL 2-deoxyglucose (2-DOG) was added to each well (Perkin-Elmer). After 5 min, 5% unlabeled glucose was added to stop the reaction. Cells were then washed two times in 5% glucose and lysed by the addition of 0.5% SDS; 3 mL Ultima Gold scintillant (Packard) were added, samples were vortexed, and radioactivity was counted.

3′ UTR Luciferase Reporter Assays

cDNA fragments corresponding to the entire 3′ UTR of carnitine O-octaniltransferase (Crot), carnitine palmitoyltransferase 1A (Cpt1a), hydroxyacyl-CoAdehydrogenase (Hadhb), AMP kinase subunit-α (Ampkα), and insulin receptor substrate 2 (Irs2), citrate synthase (Cs), 3-hydroxy-3-methylglutaryl CoA (Hmgcr), and Sirtuin 6 (Sirt6) were amplified by RT-PCR from total RNA extracted from HepG2 cells with XhoI and NotI linkers. The PCR products were directionally cloned downstream of the Renilla luciferase ORF of the ψCHECK2 vector (Promega) that also contains a constitutively expressed firefly luciferase gene, which is used to normalize transfections. Point mutations in the seed region of predicted miR-33 sites within the 3′ UTR of Crot, Cpt1a, Hadhb, Ampkα, and Sirt6 were generated using Multisite-Quickchange (Stratagene) according to the manufacturer's protocol. All constructs were confirmed by sequencing. COS-7 cells were plated into 12-well plates (Costar) and cotransfected with 1 μg indicated 3′ UTR luciferase reporter vectors and the miR-33 mimic or negative control mimic (CM) (Dharmacon) using Lipofectamine 2000 (Invitrogen). Luciferase activity was measured using the Dual-Glo Luciferase Assay System (Promega). Renilla luciferase activity was normalized to the corresponding firefly luciferase activity and plotted as a percentage of the control (cells cotransfected with the corresponding concentration of control mimic). Experiments were performed in triplicate wells of a 12-well plate and were repeated at least three times.

Antibodies

A mouse monoclonal antibody against adenosine triphosphate binding cassette 1 (ABCA1; 1:1,000) was purchased from Abcam. A mouse monoclonal antibody against IRβ (1:1,000), a goat polyclonal antibody against CPT1a (1:500), a rabbit polyclonal antibody against SIRT6 (1:500), a rabbit polyclonal antibody against IRS1 (1:1,000), a rabbit polyclonal antibody against HADHB (1:250), and a rabbit polyclonal antibody against CROT (1:250) were obtained from Novus. A mouse monoclonal antibody against HSP90 was from BD Biosciences. A rabbit polyclonal antibody against IRS2 (1:1,000) was purchased from Bethyl. A rabbit monoclonal antibody against phospho-GSK-3α/β (Ser21/9) (1:1,000), a rabbit monoclonal antibody against phospho-AKT Ser-473 (1:500), a rabbit polyclonal antibody against total AKT (1:2,000), a rabbit monoclonal antibody against phospho-ERK1,2 (1:2,000), and a mouse monoclonal antibody against ERK1,2 (1:1,000) were obtained from Cell Signaling. Secondary fluorescently labeled antibodies were from Molecular Probes (Invitrogen) and Rockland

Results

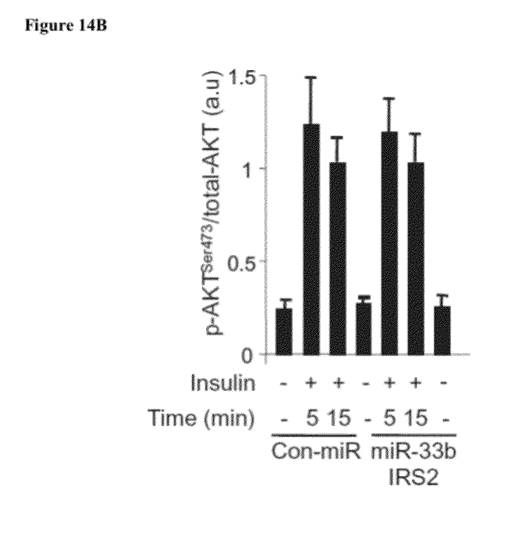

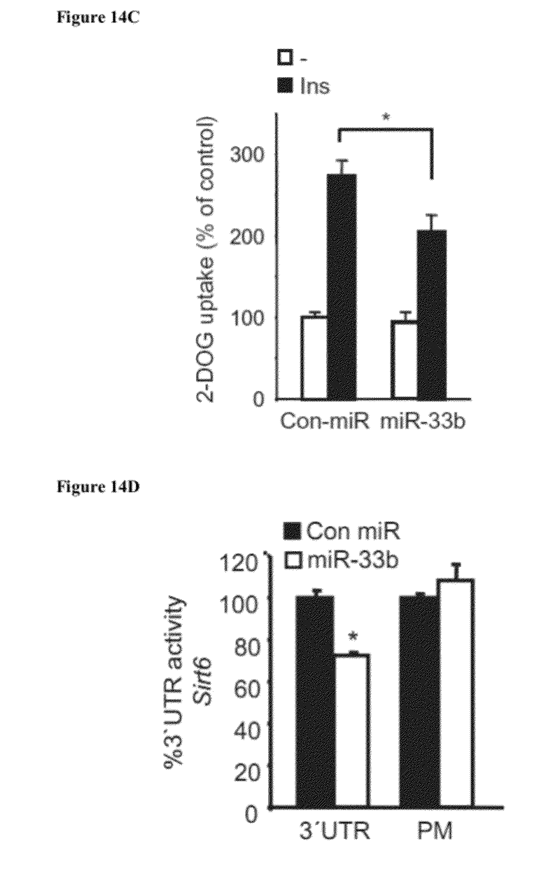

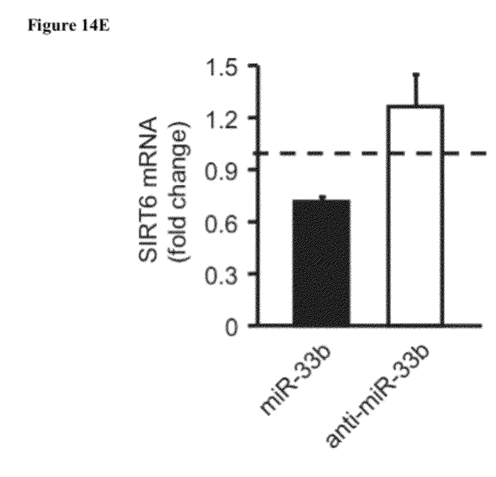

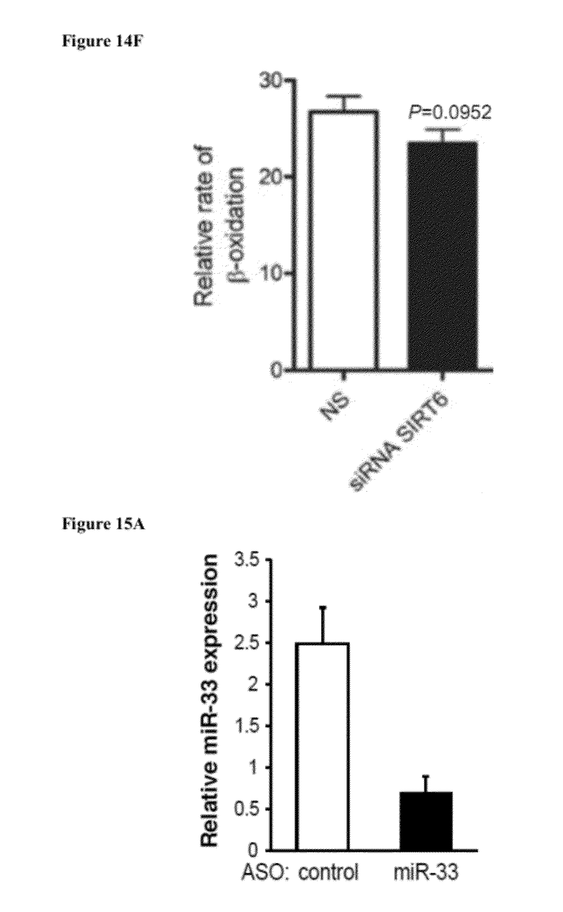

To further explore the observation that miR-33 inhibits IRS2 expression, the effect of miR-33 on insulin signaling was assessed. IRS2 is a cytoplasmic signaling molecule that mediates the effects of insulin, insulin-like growth factor 1, and other cytokines by acting as a molecular adaptor between receptor tyrosine kinases and downstream effectors. To test the role of miR-33 in regulating insulin signaling, the effect of miR-33 overexpression on two of the main downstream effectors of IRS2 (the PI3K/AKT and rat sarcoma (RAS)/RAF/ERK pathways) was analyzed. As seen in FIG. 14A, Huh7 cells transfected with miR-33b showed reduced AKT and ERK phosphorylation after insulin stimulation, indicative of reduced IRS2 function. Similar results were observed when we analyzed the AKT activation using an in vitro kinase assay. To determine whether or not IRS2 overexpression rescues the miR-33 overexpression effect on AKT phosphorylation, Huh7 cells we32 transfected with IRS2 cDNA that lacked the 3′ UTR sequence. As seen in FIG. 14B, IRS2 expression rescued the AKT activation on insulin stimulation in miR-33b-overexpressing cells.

To gain insights into the role of miR-33 in regulating insulin signaling, the effect of miR-33b on 2-deoxyglucose uptake after insulin stimulation was assessed. As seen in FIG. 14C, miR-33b overexpression reduced insulin-induced 2-deoxyglucose (2-DOG) uptake in Huh7 cells. FoxO1 cellular localization was also assessed in insulin-stimulated cells. FoxO1 is a well-defined target downstream of the conserved insulin/target of rapamycin (TOR) signaling network that has important roles in the regulation of processes as diverse as cellular growth, stress resistance, and energy homeostasis. To date, several proteins are known to interact with FoxO transcription factors, regulating their intracellular localization and/or activity. One of the best documented is the AKT/protein kinase B (PKB) kinase, which phosphorylates FoxO in three conserved sites, leading to FoxO cytoplasmatic retention and transcriptional inactivation. To test the effect of miR-33 on FoxO1 localization, Huh7 was transfected with a con-miR or miR-33b and the cells transduced with a FoxO1-GFP adenovirus. FoxO1-GFP localized primarily to the nucleus in starved cells transfected with Con-miR or miR-33b. Treatment with insulin induced FoxO1-GFP translocation from the nucleus to the cytoplasm in Con-miR-transfected cells, whereas cells transfected with miR-33b had the cellular distribution reversed. Interestingly, IRS2 overexpression rescued the effect of miR-33 overexpression on FoxO1 intracellular localization. Together, these experiments identify miR-33 as an important regulator of the insulin-signaling pathways.

In addition, the effect of miR-33b overexpression in Huh7 cells on other insulin-related genes was also assessed using an array that included 84 genes involved in carbohydrate and lipid metabolism and target genes for insulin signaling. As expected, IRS2 was down-regulated in Huh7 cells transfected with miR-33b. Moreover, glucokinase (GCK), fibroblast growth factor receptor substrate 2 (FRS2), acetyl-CoA carboxylase-α (ACACA), and peroxisome proliferator-activated-γ (PPARG) were also inhibited. Interestingly, FRS2 is also a predicted target of miR-33a and -b. FRS2 has been suggested to participate in insulin signaling by recruiting Src-homology-phosphatase 2 (SHP2) and hence, could function as a docking molecule similar to insulin receptor substrate proteins.

In addition to IRS2 and FRS2, the bioinformatic analysis identified a third miR-33 predicted target involved in glucose homeostasis: the histone deacetylase SIRT6. SIRT6-deficient mice develop normally but succumb to lethal hypoglycemia early in life, suggesting an important role of SIRT6 in regulating glucose metabolism. Interestingly, it has also been recently reported that hepatic-specific disruption of SIRT6 in mice results in fatty liver formation because of enhanced glycolysis and triglyceride synthesis. To confirm that miR-33b targets SIRT6, the Sirt6 3′ UTR was cloned into a luciferase reporter construct. miR-33b markedly repressed the 3′ UTR activity of Sirt6 (FIG. 14D), and mutation of the miR-33 target sites in the 3′ UTR relieved miR-33b repression of Sirt6, consistent with a direct interaction of miR-33b with these sites (FIG. 14D). Furthermore, miR-33b overexpression significantly inhibited the SIRT6 mRNA and protein levels in Huh7 cells (FIG. 14E), whereas inhibition of endogenous miR-33b by anti-miR-33 increased the expression of SIRT6 (FIG. 14E). Although these data are consistent with a physiological role for miR-33b in regulating SIRT6 expression, this is unlikely to contribute to the regulation of fatty acid metabolism by miR-33b, because inhibition of SIRT6 expression by siRNA only modestly decreased fatty acid β-oxidation (FIG. 14F).

### Example 9

### In Vivo Reverse Cholesterol Transport (RCT)

Materials and Methods

The effects of miR-33 inhibition on reverse cholesterol transport were evaluated in mice lacking a functional LDL receptor gene (Ldlr^{-/-} mice), which are used as a model of atherosclerosis. One group of mice (n=15) received treatment with a miR-33 antisense oligonucleotide (chemically modified and fully complementary to miR-33), and a second group (n=15) received treatment with a scrambled control antisense oligonucleotide (not complementary to miR-33).

Oligonucletides were administered to mice intraperitoneally at a frequency of 2× the first week and then weekly thereafter for a total 4 doses. Primary bone marrow derived macrophages were labeled ex vivo with ³H-cholesterol as previously described (Zhang Y, et al., Circulation, 2003, 108(6):661-3) and administered to the miR-33 antisense oligonucleotide or scrambled control treated mice 48 h before the end of the experimental time (4 weeks). The ³H tracer was measured in blood and feces after 24 and 48 hours, and in liver tissues after 48 hours.

Results

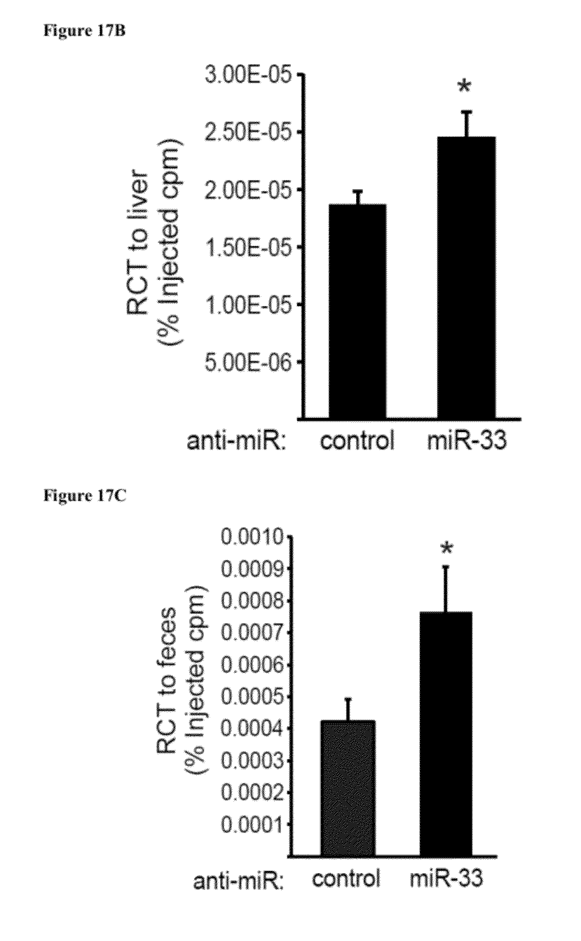

Mice treated with anti-miR-33 showed a 35% increase in reverse cholesterol transport (as measured by mobilization of ³H-cholesterol) to the serum and liver (FIGS. 17A and 17B), and a 2× increase in reverse cholesterol transport to feces (FIG. 17C) compared to control anti-miR treated mice. These results demonstrate that inhibition of miR-33 in vivo leads to increased reverse cholesterol transport and excretion of cholesterol to feces. Accordingly, miR-33 inhibitors, such as miR-33 antisense oligonucleotides, can increase reverse cholesterol transport in a subject, for example a subject who has atherosclerosis or is at risk for developing atherosclerosis.

### Example 10

### Evaluation of Anti-Atherosclerotic Effects

Materials and Methods

To test the therapeutic potential of miR-33 in a model of atherosclerosis, miR-33 was inhibited in mice lacking the LDL-receptor (Ldlr^{-/-} mice), which are used as a model of atherosclerosis.

Ldlr^{-/-} mice were fed a Western diet for 14 weeks, to establish atherosclerotic lesions (14 weeks WD feeding). After this 12 week period, mice were switched to chow diet and treated with a miR-33 antisense oligonucleotide (modified and fully complementary to miR-33) or a scrambled control oligonucleotide or PBS (control) for 4 weeks, at a frequency of 2× the first week and then weekly thereafter for a total 4 doses. Oligonucleotide was administered subcutaneously. At the end of the four week treatment period, animals were sacrificed. Blood was collected, and arteries were isolated and prepared for histological analysis.

Results

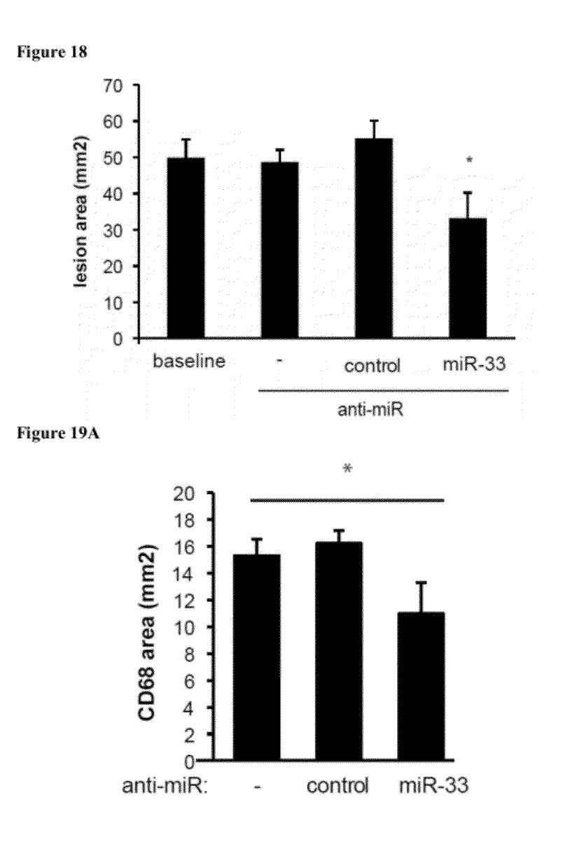

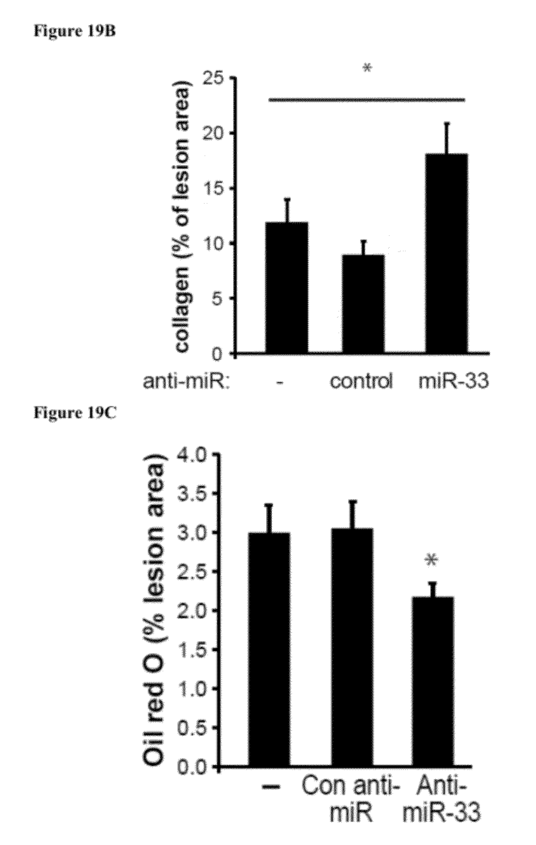

Treatment with the miR-33 antisense oligonucleotide increased circulating HDL in these hyperlipidemic mice by 36% (p≦0.05), relative to the control-treated mice. Atherosclerotic lesion areas were measured, demonstrating an approximate 35% reduction in lesion area in the aortic root in anti-miR-33 treated mice (FIG. 18). In addition, atherosclerotic lesions in anti-miR-33 treated mice showed characteristics of increased plaque stabilization including reduced CD68⁺ macrophage accumulation (FIG. 19A) and increased collagen content (FIG. 19B).

These results demonstrate that treatment with an inhibitor of miR-33 reduces atherosclerotic plaque size and favorably affects plaque characteristics (i.e., plaque stability). Accordingly, miR-33 inhibitory agents, for example miR-33 antisense oligonucleotides, can be used for the treatment of atherosclerosis, and the reduction of atherosclerotic plaque size.

### Example 11

### Evaluation of Anti-miRNA Molecule Delivery to Macrophages

Materials and Methods

Atherosclerosis Analysis

Hearts embedded in OCT were sectioned through the aortic root (8 μm), and stained with hematoxylin and eosin for lesion quantification or used for immunohistochemical analysis as previously described (Moore, K. J., et al. 2005. J Clin Invest 115:2192-2201; Manning-Tobin, J. J., et al. 2009. Arterioscler Thromb Vasc Biol 29:19-26). For morphometric analysis of lesions, 16 sections per mouse were imaged, spanning the entire aortic root, and lesions were quantified using iVision Software. For collagen analysis, 10 sections per mouse were stained with Picrosirius Red and imaged under polarized light using a Zeiss Axioplan microscope. For detection of neutral lipid, oil red O staining was performed as previously described (Moore, K. J., et al. 2005. J Clin Invest 115:2192-2201; Manning-Tobin, J. J., et al. 2009. Arterioscler Thromb Vasc Biol 29:19-26). For macrophage analysis, 10 sections per mouse were incubated with an anti-CD68 antibody (rat anti-mouse CD68, 1:500, Serotec) and a secondary antibody conjugated to biotin (1:500) and antibody reactivity was visualized using the Vectastain ABC Elite kit (Vector labs) and diaminobenzadine (DAB; Sigma). For detection of 2′F/MOE oligonucleotides frozen sections were fixed in neutral buffered formalin at room temperature, and treated with Dako Dual Endogenous Enzyme-Blocking Reagent (DAKO, Carpinteria, Calif.) for 5 minutes. Slides were rinsed in PBS and blocked with 5% normal donkey serum, followed by incubation with primary antibodies raised against the phosphorothioate backbone of the 2′F/MOE oligonucleotides or a control antibody for 1 hour. Slides were incubated with HRP conjugated donkey anti-rabbit secondary antibody for 30 minutes, and immunoreactivity was visualization with DAB substrate (DAKO).

Laser Capture Microdissection

Laser capture microdissection was performed using a PixCell II instrument (Arcturus Bioscience, Mountain View, Calif.) as previously described (69, 70). To visualize CD68-positive cells, a guide slide was prepared by staining for CD68 as described above. Cells corresponding to CD68+ area in serial sections were collected and RNA was extracted using the Arcturus Picopure RNA Isolation kit. Total RNA was amplified using the Ovation WT Pico Amp kit (NuGen), purified using Qiaquick PCR Purification kit (Qiagen) and used for quantitative PCR.

Quantitative PCR

1 μg of total RNA was reverse-transcribed using iScript cDNA Synthesis kit (BioRad), gene expression was measured by quantitative RT-PCR, and normalized to GAPDH as described (Rayner, K. J., et al. 2010. Science 328:1570-1573; Stewart, C. R., et al. 2010. Nat Immunol 11:155-161).

Results

To test whether miR-33 antisense oligonucleotides can be delivered to macrophages within an atherosclerotic plaque to directly alter target gene expression in these cells, immunohistochemical staining of aortic sinus lesions was performed to determine the localization of miR-33 antisense oligonucleotide.

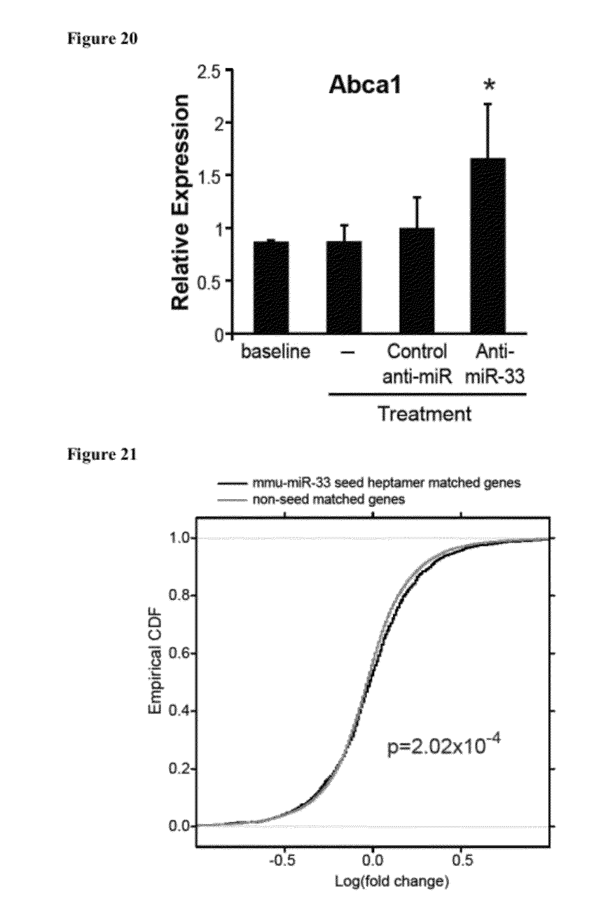

Aortic sinus lesions, isolated from the mice described in Example 8, were subjected to immunohistochemical staining using an antibody directed against the phosphorothioate backbone of the miR-33 antisense oligonucleotide. It was observed that the miR-33 antisense oligonucleotide was found within the plaque, where it co-localized with CD68-positive macrophages. To determine whether the miR-33 antisense oligonucleotide affects target gene expression within the plaque, lesional CD68+ macrophages were isolated using laser-capture microdissection and RNA was extracted for gene expression analysis. Examination of ABCA1 expression levels in lesional macrophages demonstrated similar levels of Abca1 mRNA in baseline, PBS or control anti-miR treated mice. Notably, miR-33 antisense oligonucleotide treated mice had a 66% increase in lesional macrophage ABCA1 expression compared to the control groups (FIG. 20). These results demonstrate that the miR-33 antisense oligonucleotides are capable of penetrating the atherosclerotic lesion to reach plaque macrophages, where they can directly alter target gene expression.

### Example 12

### Gene Expression Profiling in Lesional Macrophages

Materials and Methods

Affymetrix Gene Array Analysis

Messenger RNA from macrophages collected by laser capture microdissection were profiled for expression on Affymetrix Mouse 430 2.0 arrays in quadruplicates. Macrophages were derived from three groups of mice: (1) anti-miR33 treated, (2) control anti-miR treated, and (3) untreated. Microarray data was RMA normalized (Bolstad, B. M., et al. 2003. Bioinformatics 19:185-193), log2 transformed, and quality controlled by principal component analysis (PCA). The gene-level array data was then compared between treated vs untreated groups by one-way ANOVA. The genes were then split between those containing one or more mir-33 seed-matched heptamers (nucleotides 1-7 or 2-8) in their 3′UTRs and those that contained none. The R package [ref: http://www.r-project.org/] was used to compute the cumulative distribution function (CDF) for the fold changes of these genes. Statistical significance of the shift between these two populations was determined by using a 1-sided Kolmogorov-Smirnov (KS) test.

Results

To further understand the impact of the inhibition of miR-33 on plaque macrophage phenotype gene expression profiling was performed on RNA isolated from lesional macrophages using Affymetrix gene arrays. Cumulative distribution function analysis revealed that treatment with miR-33 antisense oligonucleotide resulted in a statistically significant enrichment in the expression of genes containing miR-33 binding sites in their 3′ UTR compared to non-miR33 target genes (FIG. 21; p=2.02×10⁻⁴), consistent with specific de-repression of miR-33 targets in lesional macrophages.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, equivalents to the specific embodiments of the invention described herein. Such equivalents are intended to be encompassed by the following claims.

We claim:

1. A method of treating a metabolic syndrome in a subject, comprising administering to the subject a therapeutically effective amount of a miR-33 inhibitor to treat or ameliorate one or more symptoms of a metabolic syndrome.

2. The method of claim 1, wherein the subject has elevated serum triglyceride levels, insulin resistance, non-alcoholic hepatic steatosis (fatty liver), atherosclerosis, or a combination thereof.

3. The method of claim 1, wherein the subject has normal HDL levels.

4. The method of claim 1, wherein the subject has elevated serum triglyceride levels.

5. The method of claim 4, wherein the subject has a serum triglyceride level of 150 mg/dL or greater.

6. The method of claim 1, wherein the miR-33 inhibitor is administered to a subject at risk for atherosclerosis or atherosclerotic plaque rupture.

7. The method of claim 6, wherein the miR-33 inhibitor increases cholesterol efflux in peripheral cells of the subject.

8. The method of claim 7, wherein the peripheral cell is a macrophage.

9. The method of claim 8, wherein the macrophage is present in an atherosclerotic plaque.

10. The method of claim 1, wherein the miR-33 inhibitor is an antisense oligonucleotide that is complementary to miR-33.

11. The method of claim 10, wherein the antisense oligonucleotide is complementary to at least 12 contiguous nucleotides in miR-33.

12. The method of claim 10, wherein the miR-33 is miR-33a.

13. The method of claim 12, wherein the miR-33a comprises the nucleic acid sequence SEQ ID NO:32.

14. The method of claim 10, wherein the miR-33 is miR-33b.

15. The method of claim 14, wherein the miR-33b comprises the nucleic acid sequence SEQ ID NO:34.

16. The method of claim 10, wherein the antisense oligonucleotide comprises between 7 and 25 nucleotides.

17. The method of claim 10, wherein the antisense oligonucleotide comprises between 7 and 21 nucleotides.

18. The method of claim 10, wherein the antisense oligonucleotide comprises a sequence selected from 5′-CAATGCANNNNCAATGCA-3′ (SEQ ID NO:37), 5′-CAAUGCANNNNCAAUGCA-3′ (SEQ ID NO:38), 5′-CAAUGCANNNNCAATGCA-3′ (SEQ ID NO:39), and 5′-CAATGCANNNNCAAUGCA-3′ (SEQ ID NO:40).

19. The method of claim 10, wherein one or more of the nucleotide units of the antisense oligonucleotide are locked nucleic acid (LNA) units or 2′ substituted nucleotide analogues.

20. The method of claim 10, wherein one or more of the internucleoside linkages between the nucleotide units of the antisense oligonucleotide are phosphorothioate internucleoside linkages.

21. A pharmaceutical formulation for use in the method of claim 1.

22. A miR-33 inhibitor for use in treating a metabolic syndrome in a subject.

23. The miR-33 inhibitor of claim 22, wherein the subject has elevated serum triglyceride levels, insulin resistance, non-alcoholic hepatic steatosis (fatty liver), atherosclerosis, or a combination thereof.


SEQUENCE LISTING (46 sequences)

SEQ ID NO: 1 - DNA, 28 bp, Artificial Sequence
  Synthetic Construct
ggtttggagatggttatacaatagttgt

SEQ ID NO: 2 - DNA, 17 bp, Artificial Sequence
  Synthetic Construct
cccggaaacgcaagtcc

SEQ ID NO: 3 - DNA, 22 bp, Artificial Sequence
  Synthetic Construct
tcacccagttctgcatcctctt

SEQ ID NO: 4 - DNA, 21 bp, Artificial Sequence
  Synthetic Construct
gcagatgtgtcaggaccgagt

SEQ ID NO: 5 - DNA, 20 bp, Artificial Sequence
  Synthetic Construct
ggtccgcctgtgtactttgt

SEQ ID NO: 6 - DNA, 20 bp, Artificial Sequence
  Synthetic Construct
ggcttcacccagtcgaaatg

SEQ ID NO: 7 - DNA, 20 bp, Artificial Sequence
  Synthetic Construct
aactttggcattgtggaagg

SEQ ID NO: 8 - DNA, 20 bp, Artificial Sequence
  Synthetic Construct
acacattgggggtaggaaca

SEQ ID NO: 9 - DNA, 19 bp, Artificial Sequence
  Synthetic Construct
gcgttctggagaccatgga

SEQ ID NO: 10 - DNA, 25 bp, Artificial Sequence
  Synthetic Construct
acaaagttgctctgaaaacaaatca

SEQ ID NO: 11 - DNA, 21 bp, Artificial Sequence
  Synthetic Construct
acttccctggcctatttgacc

SEQ ID NO: 12 - DNA, 20 bp, Artificial Sequence
  Synthetic Construct
ggcatggacgggtacatctt

SEQ ID NO: 13 - DNA, 22 bp, Artificial Sequence
  Synthetic Construct
cttgtggaatgccttgtgattg

SEQ ID NO: 14 - DNA, 19 bp, Artificial Sequence
  Synthetic Construct
agccgaagcagcacatgat

SEQ ID NO: 15 - DNA, 22 bp, Homo sapiens
tcaatgcaatgcaattcaatgc

SEQ ID NO: 16 - DNA, 22 bp, Homo sapiens
tctttgtaatgcaattcaatgc

SEQ ID NO: 17 - DNA, 22 bp, Homo sapiens
tctttgtaatgcaattctatgt

SEQ ID NO: 18 - DNA, 18 bp, Homo sapiens
aatcaatgcactgtctgt

SEQ ID NO: 19 - DNA, 18 bp, Homo sapiens
aatctttgcactgtctgt

SEQ ID NO: 20 - DNA, 18 bp, Mus musculus
cgcaatgcaacgcaatgc

SEQ ID NO: 21 - DNA, 18 bp, Mus musculus
cgctttgcaacgcaatgc

SEQ ID NO: 22 - DNA, 18 bp, Mus musculus
cgctttgcaacgctttgc

SEQ ID NO: 23 - RNA, 21 nt, Mus musculus
acguuacguugauguuacgug

SEQ ID NO: 24 - RNA, 23 nt, Mus musculus
uguacugauacuauucaaugcaa

SEQ ID NO: 25 - RNA, 23 nt, Mus musculus
ugacacuauucaaugcaaugcac

SEQ ID NO: 26 - RNA, 23 nt, Mus musculus
caaugcaaugcacuucaaugcaa

SEQ ID NO: 27 - RNA, 23 nt, Mus musculus
gggacuaacgcaacgcaaugcaa

SEQ ID NO: 28 - RNA, 23 nt, Mus musculus
caacgcaaugcaacgcaaugcag

SEQ ID NO: 29 - RNA, 23 nt, Mus musculus
uuaaagagcuuuauuaaugcaau

SEQ ID NO: 30 - RNA, 23 nt, Homo sapiens
ucuguaaaggccaaucaaugcac

SEQ ID NO: 31 - RNA, 23 nt, Homo sapiens
uuaaagagcuuuauuaaugcaau

SEQ ID NO: 32 - RNA, 21 nt, Homo sapiens
gugcauuguaguugcauugca

SEQ ID NO: 33 - RNA, 22 nt, Homo sapiens
caauguuuccacagugcaucac

SEQ ID NO: 34 - RNA, 20 nt, Homo sapiens
gugcauugcuguugcauugc

SEQ ID NO: 35 - RNA, 22 nt, Homo sapiens
cagugccucggcagugcagccc

SEQ ID NO: 36 - DNA, 10 bp, Artificial Sequence
  Synthetic Construct
caatgcannn

SEQ ID NO: 37 - RNA, 10 nt, Artificial Sequence
  Synthetic Construct
caaugcannn

SEQ ID NO: 38 - DNA, 18 bp, Artificial Sequence
  Synthetic Construct
caatgcannnncaatgca

SEQ ID NO: 39 - RNA, 18 nt, Artificial Sequence
  Synthetic Construct
caaugcannnncaaugca

SEQ ID NO: 40 - DNA, 18 bp, Artificial Sequence
  Synthetic Construct
caaugcannnncaatgca

SEQ ID NO: 41 - DNA, 18 bp, Artificial Sequence
  Synthetic Construct
caatgcannnncaaugca

SEQ ID NO: 42 - DNA, 21 bp, Artificial Sequence
  Synthetic Construct
tgcaatgcaactacaatgcac

SEQ ID NO: 43 - RNA, 68 nt, Homo sapiens
cuguggugcauuguaguugcauugcauguucggugguacccaugcaauguuuccacagugcaucacag

SEQ ID NO: 44 - RNA, 95 nt, Homo sapiens
gcgggcggccccgcggugcauugcuguugcauugcacguguugaggcgggugcagugccucggcagugcagcccggagccggccccuggcaccac

SEQ ID NO: 45 - DNA, 21 bp, Artificial Sequence
  Synthetic Construct
tgcaatgcaactacaatgcac

SEQ ID NO: 46 - DNA, 21 bp, Artificial Sequence
  Synthetic Construct
tccaatccaacttcaatcatc